SEQUENCE LISTING (34 sequences)

SEQ ID NO: 1 - DNA, 1314 bp, Corynebacterium glutamicum
  misc_feature 1..1314: Corynebacterium glutamicum ATCC 13032 (strain)
  CDS 1..1314: /note=“nucleotide sequence comprising locus_tag NCgl0625 disclosed in NC_003450” /transl_table=11
  misc_feature 1..1314: /gene=“metY from Corynebacterium glutamicum ATCC 13032”
atgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctag

SEQ ID NO: 2 - protein, 437 aa, Corynebacterium glutamicum
  misc_feature 1..437: Corynebacterium glutamicum ATCC 13032 (strain)
  [CDS]1..1314 from SEQ ID NO 1
MPKYDNSNADQWGFETRSIHAGQSVDAQTSARNLPIYQSTAFVFDSAEHAKQRFALEDLGPVYSRLTNPTVEALENRIASLEGGVHAVAFSSGQAATTNAILNLAGAGDHIVTSPRLYGGTETLFLITLNRLGIDVSFVENPDDPESWQAAVQPNTKAFFGETFANPQADVLDIPAVAEVAHRNSVPLIIDNTIATAALVRPLELGADVVVASLTKFYTGNGSGLGGVLIDGGKFDWTVEKDGKPVFPYFVTPDAAYHGLKYADLGAPAFGLKVRVGLLRDTGSTLSAFNAWAAVQGIDTLSLRLERHNENAIKVAEFLNNHEKVEKVNFAGLKDSPWYATKEKLGLKYTGSVLTFEIKGGKDEAWAFIDALKLHSNLANIGDVRSLVVHPATTTHSQSDEAGLARAGVTQSTVRLSVGIETIDDIIADLEGGFAAI

SEQ ID NO: 3 - DNA, 1331 bp, Corynebacterium glutamicum
  misc_feature 1..1331: Corynebacterium glutamicum ATCC 13032 (strain)
  misc_feature 4..9: /note=“restriction site NdeI”
  CDS 7..1320: /transl_table=11
  misc_feature 1321..1328: /note=“restriction site NotI”
aaacatatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctaggcggccgcaaa

SEQ ID NO: 4 - protein, 437 aa, Corynebacterium glutamicum; identical to SEQ ID NO: 2
  misc_feature 1..437: Corynebacterium glutamicum ATCC 13032 (strain)
  [CDS]7..1320 from SEQ ID NO 3

SEQ ID NO: 5 - DNA, 1314 bp, Corynebacterium glutamicum
  misc_feature 1..1314: Corynebacterium glutamicum ATCC 13032 (strain)
  CDS 1..1314: /transl_table=11
  misc_feature 1..1314: /gene=“metY allelic varation from Corynebacterium glutamicum ATCC 13032”
  misc_feature 4..6: /note=“aca codon for threonine at amino acid position 2”
atgacaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctag

SEQ ID NO: 6 - protein, 437 aa, Corynebacterium glutamicum
  misc_feature 1..437: Corynebacterium glutamicum ATCC 13032 (strain)
  [CDS]1..1314 from SEQ ID NO 5
MTKYDNSNADQWGFETRSIHAGQSVDAQTSARNLPIYQSTAFVFDSAEHAKQRFALEDLGPVYSRLTNPTVEALENRIASLEGGVHAVAFSSGQAATTNAILNLAGAGDHIVTSPRLYGGTETLFLITLNRLGIDVSFVENPDDPESWQAAVQPNTKAFFGETFANPQADVLDIPAVAEVAHRNSVPLIIDNTIATAALVRPLELGADVVVASLTKFYTGNGSGLGGVLIDGGKFDWTVEKDGKPVFPYFVTPDAAYHGLKYADLGAPAFGLKVRVGLLRDTGSTLSAFNAWAAVQGIDTLSLRLERHNENAIKVAEFLNNHEKVEKVNFAGLKDSPWYATKEKLGLKYTGSVLTFEIKGGKDEAWAFIDALKLHSNLANIGDVRSLVVHPATTTHSQSDEAGLARAGVTQSTVRLSVGIETIDDIIADLEGGFAAI

SEQ ID NO: 7 - DNA, 1331 bp, Corynebacterium glutamicum
  misc_feature 1..1331: Corynebacterium glutamicum ATCC 13032 (strain)
  misc_feature 4..9: /note=“restriction site NdeI”
  CDS 7..1320: /transl_table=11
  misc_feature 10..12: /note=“aca codon for threonine at amino acid position 2”
  misc_feature 1321..1328: /note=“restriction site NotI”
aaacatatgacaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctaggcggccgcaaa

SEQ ID NO: 8 - protein, 437 aa, Corynebacterium glutamicum; identical to SEQ ID NO: 6
  misc_feature 1..437: Corynebacterium glutamicum ATCC 13032 (strain)
  [CDS]7..1320 from SEQ ID NO 7

SEQ ID NO: 9 - DNA, 1185 bp, Chromobacterium violaceum
  misc_feature 1..1185: Chromobacterium violaceum ATCC 12472 (strain)
  CDS 1..1185: /note=“nucleotide sequence comprising locus_tag CV_2725 disclosed in AE016825” /transl_table=11
  misc_feature 1..1185: /gene=“metZ from Chromobacterium violaceaum ATCC 12472”
atggcatccgacgcgccgcatcttccgctgcaccctgaaaccctggccatccgggccgggttggaaaccagccagttcaacgagcacagccagggcctgttcctgacgtccagcttcacctacgaatcggccgcgcaggcggcggcgatgttcctgggcgagatcgacggctacacctattcccgcttcaccaatccgaccgtcgccgcgttccagcataggctggcgcagatggagggcggggagcgcgccatcgccaccgccaccggcatggcggcgatccaggccatcatgatgactttgctgcaggctggcgaccacatcgtgtcgtcgcaaagcctgttcggctccaccaccaatctgttcgccaaccagttggccaagttcgccgtggccaccgacttcgtcgacgcgcgcgacctgtccgcctggcgggaggcgctgcggccgaacaccaagctgctgttcctggagacgccgtccaatcccttgaccgaagtggccgacatcgcggccatcgccgacatcgcccacgcgcatggcgcgctgctggtggtggacaacagcttctgttcgccggccttgcagcagccgttgaaactgggcgccgatctggtcatgcattccgccaccaagttcatcgacggccatggccgggtgatgggcggggcggtggtcggcagcgacaagctggtcgagcaggtctatttgcacgtgcgcgccgccggtccctcgctggcgccgttcaatgcctggacgctgctgtccggtttggagacgctgcacctgcggatggagaagcacagcgccaacgcgctggagctggcgcgctggctggaggcgcagcccaatgtggagcgcgtctattacccgggcctggagagccacccccagcacgagctggcgctgcgccagcagaagagcggcggagcggtggtgtccttcgtggtcaagggcggccgcaaggccgcgtggaaagtggtggacgcggtcagggtgatctcgcgcaccgccaatctgggcgatgtgaaaaccaccctcactcatccggccagcaccacccacgcccgcgtgacgcaggaggcgcgcgagcgcgccggcatcgtcgaggggctgttgcgcgtcagcgtcggcctggaaaatgtacgggaccttcaacaagatctgttgcggggccttgactaa

SEQ ID NO: 10 - protein, 394 aa, Chromobacterium violaceum
  misc_feature 1..394: Chromobacterium violaceum ATCC 12472 (strain)
  [CDS]1..1185 from SEQ ID NO 9
MASDAPHLPLHPETLAIRAGLETSQFNEHSQGLFLTSSFTYESAAQAAAMFLGEIDGYTYSRFTNPTVAAFQHRLAQMEGGERAIATATGMAAIQAIMMTLLQAGDHIVSSQSLFGSTTNLFANQLAKFAVATDFVDARDLSAWREALRPNTKLLFLETPSNPLTEVADIAAIADIAHAHGALLVVDNSFCSPALQQPLKLGADLVMHSATKFIDGHGRVMGGAVVGSDKLVEQVYLHVRAAGPSLAPFNAWTLLSGLETLHLRMEKHSANALELARWLEAQPNVERVYYPGLESHPQHELALRQQKSGGAVVSFVVKGGRKAAWKVVDAVRVISRTANLGDVKTTLTHPASTTHARVTQEARERAGIVEGLLRVSVGLENVRDLQQDLLRGLD

SEQ ID NO: 11 - DNA, 1200 bp, Chromobacterium violaceum
  misc_feature 1..1200: Chromobacterium violaceum ATCC 12472 (strain)
  misc_feature 4..9: /note=“restriction site NdeI”
  CDS 7..1191: /transl_table=11
  misc_feature 1191..1197: /note=“restriction site XhoI”
aaacatatggcatccgacgcgccgcatcttccgctgcaccctgaaaccctggccatccgggccgggttggaaaccagccagttcaacgagcacagccagggcctgttcctgacgtccagcttcacctacgaatcggccgcgcaggcggcggcgatgttcctgggcgagatcgacggctacacctattcccgcttcaccaatccgaccgtcgccgcgttccagcataggctggcgcagatggagggcggggagcgcgccatcgccaccgccaccggcatggcggcgatccaggccatcatgatgactttgctgcaggctggcgaccacatcgtgtcgtcgcaaagcctgttcggctccaccaccaatctgttcgccaaccagttggccaagttcgccgtggccaccgacttcgtcgacgcgcgcgacctgtccgcctggcgggaggcgctgcggccgaacaccaagctgctgttcctggagacgccgtccaatcccttgaccgaagtggccgacatcgcggccatcgccgacatcgcccacgcgcatggcgcgctgctggtggtggacaacagcttctgttcgccggccttgcagcagccgttgaaactgggcgccgatctggtcatgcattccgccaccaagttcatcgacggccatggccgggtgatgggcggggcggtggtcggcagcgacaagctggtcgagcaggtctatttgcacgtgcgcgccgccggtccctcgctggcgccgttcaatgcctggacgctgctgtccggtttggagacgctgcacctgcggatggagaagcacagcgccaacgcgctggagctggcgcgctggctggaggcgcagcccaatgtggagcgcgtctattacccgggcctggagagccacccccagcacgagctggcgctgcgccagcagaagagcggcggagcggtggtgtccttcgtggtcaagggcggccgcaaggccgcgtggaaagtggtggacgcggtcagggtgatctcgcgcaccgccaatctgggcgatgtgaaaaccaccctcactcatccggccagcaccacccacgcccgcgtgacgcaggaggcgcgcgagcgcgccggcatcgtcgaggggctgttgcgcgtcagcgtcggcctggaaaatgtacgggaccttcaacaagatctgttgcggggccttgactaactcgagaaa

SEQ ID NO: 12 - protein, 394 aa, Chromobacterium violaceum; identical to SEQ ID NO: 10
  misc_feature 1..394: Chromobacterium violaceum ATCC 12472 (strain)
  [CDS]7..1191 from SEQ ID NO 11

SEQ ID NO: 13 - DNA, 1200 bp, Hyphomonas neptunium
  misc_feature 1..1200: Hyphomonas neptunium ATCC 15444 (strain)
  CDS 1..1200: /note=“nucleotide sequence comprising locus_tag HNE_2672 disclosed in CP000158” /transl_table=11
  misc_feature 1..1200: /gene=“metZ from Hyphomonas neptunium ATCC 15444”
atggcggatgcacccggcggcgacaagaagggctggaagcctgcgacccaggcggtacgcggcggcctgatgcggtcccagcatggggagatttccgaggcgctgtatctgacctccggctacgcttacgactcggccgagcaggcgatgcgccggatggcgggcgaggaagaaggcttcgtctattcccgctatggcagcccgaccaatgagatgctgcaacagcgcctcgcgctgattgaaggcgccgaagcgtgccgggtgacgggctctggcatgggcgcgatttcgtcggccatcctggcgccgcttaaagcgggcgaccgggtggtggcggcgaccgcgctgtttggctcgtgccgctggatcattgccaaccagatgccgaagtttggcatcgaggcagtgttcgtggacggggccgatcttgatgcttggaagcgcgagatcgacaagggctgccagctggtgctgatcgaaagcccggccaatccgttgctcgacggcgtggacatcgaagcggtcgccaggctcgccaaggcggcgggcgcgctgctggtggtggacaatgtgtttgccacgccggtgcttcagcggccgctggaaatgggcgccgatgtgatcgcctattcggccaccaaacatatggacgggcagggccgcgttctgctgggcgcgatcctgacggacgccaagcggatgagtgatgtgtatgatccgtggctgcgccatatggggccggcggcctcgccgtttaacgcctgggtagtgctgaagggccttgagacgatgcagctgcgcgtggaagcgcagagccgcacggcggcgcggctggcggatgttctggccgatcatccggcggtcaatgccgtgcgctatccccaccgcaaggatcacccgcattatgaggtgcacaagcgccagatgaaatcgggcggcacgctgctcgcgctgtcgctcaagggcgggcaggacgcggcgttccgcttcctcaacgggctgcagctggtcgacatctgcaacaaccttggcgatacgaaatcgctggcctgtcatccctccaccacgacgcaccgcgcgttgagtgatgaggatcaggcggcgatggggcttgaccgcagctgggtccggctctctgttggtcttgaagacgcagatgatctggaagctgatcttctcgcttcgcttaacagcttgtga

SEQ ID NO: 14 - protein, 399 aa, Hyphomonas neptunium
  misc_feature 1..399: Hyphomonas neptunium ATCC 15444 (strain)
  [CDS]1..1200 from SEQ ID NO 13
MADAPGGDKKGWKPATQAVRGGLMRSQHGEISEALYLTSGYAYDSAEQAMRRMAGEEEGFVYSRYGSPTNEMLQQRLALIEGAEACRVTGSGMGAISSAILAPLKAGDRVVAATALFGSCRWIIANQMPKFGIEAVFVDGADLDAWKREIDKGCQLVLIESPANPLLDGVDIEAVARLAKAAGALLVVDNVFATPVLQRPLEMGADVIAYSATKHMDGQGRVLLGAILTDAKRMSDVYDPWLRHMGPAASPFNAWVVLKGLETMQLRVEAQSRTAARLADVLADHPAVNAVRYPHRKDHPHYEVHKRQMKSGGTLLALSLKGGQDAAFRFLNGLQLVDICNNLGDTKSLACHPSTTTHRALSDEDQAAMGLDRSWVRLSVGLEDADDLEADLLASLNSL

SEQ ID NO: 15 - DNA, 1254 bp, Hyphomonas neptunium
  misc_feature 1..1254: Hyphomonas neptunium ATCC 15444 (strain)
  misc_feature 4..9: /note=“restriction site XbaI”
  CDS 46..1245: /transl_table=11
  misc_feature 1246..1251: /note=“restriction site XhoI”
aaatctagaaataattttgtttaactttaagaaggagatatacatatggcggatgcacccggcggcgacaagaagggctggaagcctgcgacccaggcggtacgcggcggcctgatgcggtcccagcatggggagatttccgaggcgctgtatctgacctccggctacgcttacgactcggccgagcaggcgatgcgccggatggcgggcgaggaagaaggcttcgtctattcccgctatggcagcccgaccaatgagatgctgcaacagcgcctcgcgctgattgaaggcgccgaagcgtgccgggtgacgggctctggcatgggcgcgatttcgtcggccatcctggcgccgcttaaagcgggcgaccgggtggtggcggcgaccgcgctgtttggctcgtgccgctggatcattgccaaccagatgccgaagtttggcatcgaggcagtgttcgtggacggggccgatcttgatgcttggaagcgcgagatcgacaagggctgccagctggtgctgatcgaaagcccggccaatccgttgctcgacggcgtggacatcgaagcggtcgccaggctcgccaaggcggcgggcgcgctgctggtggtggacaatgtgtttgccacgccggtgcttcagcggccgctggaaatgggcgccgatgtgatcgcctattcggccaccaaacatatggacgggcagggccgcgttctgctgggcgcgatcctgacggacgccaagcggatgagtgatgtgtatgatccgtggctgcgccatatggggccggcggcctcgccgtttaacgcctgggtagtgctgaagggccttgagacgatgcagctgcgcgtggaagcgcagagccgcacggcggcgcggctggcggatgttctggccgatcatccggcggtcaatgccgtgcgctatccccaccgcaaggatcacccgcattatgaggtgcacaagcgccagatgaaatcgggcggcacgctgctcgcgctgtcgctcaagggcgggcaggacgcggcgttccgcttcctcaacgggctgcagctggtcgacatctgcaacaaccttggcgatacgaaatcgctggcctgtcatccctccaccacgacgcaccgcgcgttgagtgatgaggatcaggcggcgatggggcttgaccgcagctgggtccggctctctgttggtcttgaagacgcagatgatctggaagctgatcttctcgcttcgcttaacagcttgtgactcgagaaa

SEQ ID NO: 16 - protein, 399 aa, Hyphomonas neptunium; identical to SEQ ID NO: 14
  misc_feature 1..399: Hyphomonas neptunium ATCC 15444 (strain)
  [CDS]46..1245 from SEQ ID NO 15

SEQ ID NO: 17 - protein, 386 aa, Escherichia coli
  misc_feature 1..386: Escherichia coli K-12 (strain)
  metB
MTRKQATIAVRSGLNDDEQYGCVVPPIHLSSTYNFTGFNEPRAHDYSRRGNPTRDVVQRALAELEGGAGAVLTNTGMSAIHLVTTVFLKPGDLLVAPHDCYGGSYRLFDSLAKRGCYRVLFVDQGDEQALRAALAEKPKLVLVESPSNPLLRVVDIAKICHLAREVGAVSVVDNTFLSPALQNPLALGADLVLHSCTKYLNGHSDVVAGVVIAKDPDVVTELAWWANNIGVTGGAFDSYLLLRGLRTLVPRMELAQRNAQAIVKYLQTQPLVKKLYHPSLPENQGHEIAARQQKGFGAMLSFELDGDEQTLRRFLGGLSLFTLAESLGGVESLISHAATMTHAGMAPEARAAAGISETLLRISTGIEDGEDLIADLENGFRAANKG

SEQ ID NO: 18 - protein, 403 aa, Pseudomonas aeruginosa
  misc_feature 1..403: Pseudomonas aeruginosa PAO1 (strain)
  metZ
MTQDWDAGRLDSDLEGAAFDTLAVRAGQRRTPEGEHGEALFTTSSYVFRTAADAAARFAGEVPGNVYSRYTNPTVRTFEERIAALEGAEQAVATASGMSAILALVMSLCSSGDHVLVSRSVFGSTISLFDKYFKRFGIQVDYPPLSDLAAWEAACKPNTKLFFVESPSNPLAELVDIAALAEIAHAKGALLAVDNCFCTPALQQPLKLGADVVIHSATKYIDGQGRGMGGVVAGRGEQMKEVVGFLRTAGPTLSPFNAWLFLKGLETLRIRMQAHSASALALAEWLERQPGIERVYYAGLPSHPQHELARRQQSGFGAVVSFDVKGGRDAAWRFIDATRMVSITTNLGDTKTTIAHPATTSHGRLSPEDRARAGIGDSLIRVAVGLEDLDDLKADMARGLAAL

SEQ ID NO: 19 - protein, 425 aa, Pseudomonas aeruginosa
  misc_feature 1..425: Pseudomonas aeruginosa PAO1 (strain)
  metY
MKLETLAVHAGYSPDPTTRAVAVPIYQTTSYAFDDTQHGADLFDLKVPGNIYTRIMNPTNDVLEQRVAALEGGVGALAVASGMAAITYAIQTVAEAGDNIVSVAKLYGGTYNLLAHTLPRIGIQARFAAHDDVAALEALIDERTKAVFCETIGNPAGNIIDLQALADAAHRHGVPLIVDNTVATPVLCRPFEHGADIVVHSLTKYMGGHGTSIGGIVVDSGKFDWAANKSRFPLLNTPDPSYHGVTYTEAFGPAAFIGRCRVVPLRNMGAALSPFNAFLILQGLETLALRMERHCDNALAVARYLQQHPQVAWVKYAGLADNPEHALARRYLGGRPAAILSFGIQGGSAAGARFIDALKLVVRLVNIGDAKSLACHPASTTHRQLNAEELARAGVSDDMVRLSIGIEHIDDILADLDQALAAAAR

SEQ ID NO: 20 - protein, 403 aa, Pseudomonas putida
  misc_feature 1..403: Pseudomonas putida KT2440 (strain)
  metZ
MTDQWDAGRLDSDLEGVGFDTLAVRAGQNRTPEGEHSEALFLTSSYVFRTAADAAARFAGETPGNVYSRYTNPSVRAFEERLAAMEGAEQAVGTSTGMAAILAVVMSLCSAGDHVLVSQSVFGSTISLFEKYFKRFGVEVDYVPLVDLTGWEKAIKANTKLLIVESPSNPLAELVDITALSEIAHAQGAMLVVDNCFSTPALQQPLKLGADIVFHSATKFIDGQGRCMGGVVAGRTEQMKEVVGFLRTAGPTLSPFNAWIFTKGLETLRLRMRAHCESAQALAEWLEQQDGVEKVHYAGLPSHPQHELAKRQMSGFGAVVSFEVKGGKEGAWRFIDATRVISITTNLGDSKTTIAHPATTSHGRLSPQEREAAGIRDSLIRVAVGLEDVADLQADLARGLAAL

SEQ ID NO: 21 - protein, 386 aa, Corynebacterium glutamicum
  misc_feature 1..386: Corynebacterium glutamicum ATCC 13032 (strain)
  metB
MSFDPNTQGFSTASIHAGYEPDDYYGSINTPIYASTTFAQNAPNELRKGYEYTRVGNPTIVALEQTVAALEGAKYGRAFSSGMAATDILFRIILKPGDHIVLGNDAYGGTYRLIDTVFTAWGVEYTVVDTSVVEEVKAAIKDNTKLIWVETPTNPALGITDIEAVAKLTEGTNAKLVVDNTFASPYLQQPLKLGAHAVLHSTTKYIGGHSDVVGGLVVTNDQEMDEELLFMQGGIGPIPSVFDAYLTARGLKTLAVRMDRHCDNAEKIAEFLDSRPEVSTVLYPGLKNHPGHEVAAKQMKRFGGMISVRFAGGEEAAKKFCTSTKLICLAESLGGVESLLEHPATMTHQSAAGSQLEVPRDLVRISIGIEDIEDLLADVEQALNNL

SEQ ID NO: 22 - protein, 434 aa, Leptospira interrogans
  misc_feature 1..434: Leptospira interrogans serovar Lai str. 56601 (strain)
  met17
MPRNYKPETIALHGGQSPDPSTLSRAVPIYQTTSYVFKNTEHAAKLFGLQEFGNIYTRIMNPTTDVLEQRIAALEGGVAALATASGQAAETLALLNIVEAGQEIVASSSLYGGTYNLLHYTFPKLGIKVHFVDPSDPENFRKAVNDKTRAFYAETLGNPKLNTLNLEAIAKVAHDSEVPLIIDNTLPSPYLVNPIEHGADIVVHSLTKFLGGHGTSIGGIIVDSGKFNWGNGKFKNFTEPDPSYHGLKFWEVFGKFEPFGGVNIAYIIKAKVQGLRDMGASISPFNAWQILQGVETLPLRMRKHSENALAVAEYLTKHPKVSWVNYPGLKMDKNYSLAKKYHKKDLYGAILGFGIKGGAVEAKKFIDGLELFSLLANVGDAKSLVIHPASTTHQQLTPEEQLSAGVTPDFVRLSVGLENIEDILFDLEEALKKV

SEQ ID NO: 23 - protein, 444 aa, Saccharomyces cerevisiae
  misc_feature 1..444: Saccharomyces cerevisiae S288c (strain)
  Met17
MPSHFDTVQLHAGQENPGDNAHRSRAVPIYATTSYVFENSKHGSQLFGLEVPGYVYSRFQNPTSNVLEERIAALEGGAAALAVSSGQAAQTLAIQGLAHTGDNIVSTSYLYGGTYNQFKISFKRFGIEARFVEGDNPEEFEKVFDERTKAVYLETIGNPKYNVPDFEKIVAIAHKHGIPVVVDNTFGAGGYFCQPIKYGADIVTHSATKWIGGHGTTIGGIIVDSGKFPWKDYPEKFPQFSQPAEGYHGTIYNEAYGNLAYIVHVRTELLRDLGPLMNPFASFLLLQGVETLSLRAERHGENALKLAKWLEQSPYVSWVSYPGLASHSHHENAKKYLSNGFGGVLSFGVKDLPNADKETDPFKLSGAQVVDNLKLASNLANVGDAKTLVIAPYFTTHKQLNDKEKLASGVTKDLIRVSVGIEFIDDIIADFQQSFETVFAGQKP

SEQ ID NO: 24 - protein, 404 aa, Nocardia farcinica
  misc_feature 1..404: Nocardia farcinica IFM 10152 (strain)
  metZ
MITGGAFDKPLPEGVGPATLGVRGGLRRSGFEETAEALYLTSGFVYESAEAAEAAFTGEVEHFVYSRYGNPTVAMFEERIRLMDGAEAAFATASGMSAVFTALGALLGAGDRLVAARSLFGSCFVVCNEILPRWGVETVFVDGEDLDQWERALSVPTAAVFFETPANPMQTLVDVRRVTELAHAAGAKVVLDNVFATPLLQKGFDLGADVVVYSGTKHIDGQGRVLGGAILGDREYIDGPVKTLMRHTGPALSPFNAWTLLKGLETMPLRVRHSTESALRIARFLESNPAVSWVKYPFLESHPQYDLARAQMSGGGTVVTFELKAAEGEAKKRAFEVLDRLRIIDISNNLGDAKTLITHPATTTHRAMGPEGRAGIGLTDGVVRISVGLEDVDDLLSDLEHALS

SEQ ID NO: 25 - protein, 395 aa, Bradyrhizobium japonicum
  misc_feature 1..395: Bradyrhizobium japonicum USDA 6 (strain)
  metZ
MSKSTANYRPETRLVHSGTLRSQYGETSEALFLTQGYVYNSAEECEARFKGEDPGFIYSRYSNPTIAMFERRMIELEGAEAARSAATGMAAVTTAILAPLKAGDHVVASRALFGSCLYVIQDLLPRYGIETTLVDGLDLDQWQRALRPNTKTFFLESPTNPTLDVLDIPGIAEIAHSGGARLVVDNVFATPIWQSPLALGADVVVYSATKHIDGQGRCLGGIILSSEAFIAEHIHNFMRQTGPSISPFNAWVLLKGLETLGVRVRAQTETAGRIADVLASHPKISRLVYPGRADHPQAALVKKQMRGGSTLVGFEVKGGKQGAFRVLNELKLAKISNNLGDAKSLVTHPATTTHQRLKPEDRAALGISEGFIRFSAGLEHADDLIEDLTAALEKA

SEQ ID NO: 26 - protein, 383 aa, Methylococcus capsulatus
  misc_feature 1..383: Methylococcus capsulatus str. Bath (strain)
  metZ
METRAVRAGQRRTMEQEHAEPIFATSSYVFASAAEAAERFAGKAAGNIYSRFTNPTVRTFEERLAALEGGERCVAVGSGMAAIASTAFGLLKAGDHVVCSRSVFGNTTLLFQNYLAKFGVPTTFVGLTDYDGWAAAIRPETRFLFIETPSNPLTEIADIPRLAEIAHSRGCLLVVDNCFCTPALQRPLALGADIVIHSATKYLDGQGRCVGGAIVGGRELLDAEIYPFLRTGGPSMSPFNAWVFLKGLETLNLRMKAHCENALGLARWLEAQPWVERVHYPGLASHPQHELAARQQSGFGGIVSFEVKGGQEAAWRLIDSTRLLSITGNLGDAKTTITHPATTTHGRLSPEARAAAGIADGLIRIAVGLENLADIQADLARFA

SEQ ID NO: 27 - protein, 392 aa, Methylobacillus flagellatus
  misc_feature 1..392: Methylobacillus flagellatus KT (strain)
  MFLA_RS08660
MSQHEWHAETLGVRAGSEHTPFGENSEAMFLTSSFVFENAAQAAARFGGQEPGNIYSRFTNPTVSMFQNKLAALEGAEFCVATSSGMSAILACVMGVCSAGDHVVASRSIFGTSVQLFSNILKRWGLETTFVQLSDPEAWTAAVKPNTKLFFLETPSNPLTEICDIAVVAEIAHQAGALLAVDNCFCTPALQKPLALGADIVVHSATKYIDGQGRCLGGAVLGRKDVLEPVYGFLRTAGPTMSAFNAWVFLKGLETLHLRMEAHARNALALAQWLEQQPRVERVYYPGLPSHPQYALAQKQQKSGGAIVSFDVKGGQPAAWHLIDATRMLSITANLGDAKSTITHPATTTHSRVSAEARAAAGIGDGLVRIAVGLEHIDDIKADLAWLGHQD

SEQ ID NO: 28 - protein, 391 aa, Nitrosomonas europaea
  misc_feature 1..391: Nitrosomonas europaea ATCC 19718 (strain)
  metZ
MTNDLDPETLAIHTGVHRSQFNEHSESLYLTSSFVFDSAAQAAARFSGQEPGNIYSRFTNPTVTAMQERLAVLEGAEACIATASGMSAILTCVMGLLSAGDHIVASRSLFGSTVSLFNNILSRFGIQTTFVSATDPAEWQAAVRPNTRLFFLETPSNPLTEISDIAALAEIAKRAGVWLAVDNCFCTPIIQQPLKLGADLVIHSATKYLDGQGRVLGGAILGKRDLLMDSGIFSFLRTAGPSLSAFNAWIILKGMETLSLRVKAHSDHALEVARWLETHPRVGRVFYPGLPSHPQHELAMRQQKTGGGIVSFEVKGGREAAWRVVDAARLMSITANLGDTKSTLTHPATTTHGRISQEAREAAGIRDGLLRIAVGLESPDDLKADLARGLQ

SEQ ID NO: 29 - protein, 386 aa, Klebsiella pneumoniae
  misc_feature 1..386: Klebsiella pneumoniae subsp. pneumoniae MGH 78578 (strain)
  metB
MTRKQATIAVRSGLNDDEQYGCVVPPIHLSSTYNFTGFNEPRAHDYSRRGNPTRDVVQRALAELEGGAGAVLTNTGMSAILLVTTVFLKPGDLLVAPHDCYGGSYRLFDSLAKRGCYRVQFVDQSDEQALRAALAEKPKLVLVESPSNPLLRVVDIAKICGLAREAGAISVVDNTFLSPALQNPLALGADLVLHSCTKYLNGHSDVVAGVVIAKDPATVTELAWWANNIGVTGSAFDSYLLLRGLRTLSPRMEVAQRNALAIVEYLKTQPLVKKLYHPSLPENQGHEIAARQQKGFGAMLSFELDGDEQTLRRFLSGLSLFTLAESLGGVESLISHAATMTHAGMAPEARAAAGISETLLRISTGIEDGEDLIADLENGFRAANEE

SEQ ID NO: 30 - protein, 373 aa, Bacillus subtilis
  misc_feature 1..373: Bacillus subtilis subsp. subtilis str. 168 (strain)
  metI
MSQHVETKLAQIGNRSDEVTGTVSAPIYLSTAYRHRGIGESTGFDYVRTKNPTRQLVEDAIANLENGARGLAFSSGMAAIQTIMALFKSGDELIVSSDLYGGTYRLFENEWKKYGLTFHYDDFSDEDCLRSKITPNTKAVFVETPTNPLMQEADIEHIARITKEHGLLLIVDNTFYTPVLQRPLELGADIVIHSATKYLGGHNDLLAGLVVVKDERLGEEMFQHQNAIGAVLPPFDSWLLMRGMKTLSLRMRQHQANAQELAAFLEEQEEISDVLYPGKGGMLSFRLQKEEWVNPFLKALKTICFAESLGGVESFITYPATQTHMDIPEEIRIANGVCNRLLRFSVGIEHAEDLKEDLKQALCQVKEGAVSFE

SEQ ID NO: 31 - protein, 386 aa, Shigella flexneri
  misc_feature 1..386: Shigella flexneri 2a str. 2457T (strain)
  metB
MTRKQATIAVRSGLNDDEQYGCVVPPIHLSSTYNFTGFNEPRAHDYSRRGNPTRDVVQRALAELEGGAGAVLTNTGMSAIHLVTTVFLKPGDLLVAPHDCYGGSYRLFDSLAKRGCYRVLFVDQGDEQALRAALAEKPKLVLVESPSNPLLRVVDIAKICHLAREVGAVSVVDNTFLSPALQNPLALGADLVLHSCTKYLNGHSDVVAGVVIAKDPDVVTELAWWANNIGVTGGAFDSYLLLRGLRTLVPRMELAQRNAQAIVKYLQTQPLVKKLYHPSLPENQGHEIAARQQKGFGAMLSFELDGDEQTLCRFLGGLSLFTLAESLGGVESLISHAATMTHAGMAPEARAAAGISETLLRISTGIEDGEDLIADLENGFRAANKG

SEQ ID NO: 32 - protein, 388 aa, Colwellia psychrerythraea
  misc_feature 1..388: Colwellia psychrerythraea 34H (strain)
  metB
MSITKKGNITTSAVRAGINTDQQHGAVVAPIYLSSTYSLKGFNNKRQFDYSRTGNPTRATFAGAIAELEQGSVGIVTSTGMAAVHLICQLLSTQDTVVIPHDCYGGSFRLFTHLAKRGQFKLIVVDQNDQQALDNALAHKPKLVLLESPSNPLLRLVDIEVVTKACHAVGALVAVDNTFLSPALQQPLTLGADIVFHSTTKYINGHSDVVGGVVVAKTEELGEQLAWWANCIGITGSAFDSFLALRGLKTLPVRMKQHQENALRVADFLKNHDAIDAIYFPGFPEHTGHHIAKKQQYGFGAMLSFEIKGDVEAVKKLFENLELFTLAQSLGGVESLISHPSTMTHAGMTIPDQLEAGITQSLVRISVGIEDIDDILADLAHGLTQSQL

SEQ ID NO: 33 - protein, 386 aa, Salmonella enterica
  misc_feature 1..386: Salmonella enterica subsp. enterica serovar Paratyphi A str. ATCC 9150 (strain)
  metB
MTRKQATIAVRSGLNDDEQYGCVVPPIHLSSTYNFTGFNEPRAHDYSRRGNPTRDVVQRALAELEGGAGAVLTNTGMSAIHLVTTVFLKPGDLLVAPHDCYGGSYRLFDSLATRGCYCVRFVDQGDERALQAALEEKPKLVLVESPSNPLLRVVDIAKICRLAREAGAVSVVDNTFLSPALQNPLALGADLVLHSCTKYLNGHSDVVAGVVIAKDPEVVTELAWWANNIGVTGGAFDSYLLLRGLRTLVPRMELAQRNAQAIVKYLQTQPLVKKLYHPSLPENQGHEIAARQQKGFGAMLSFELDGDEETLRRFLGGLSLFTLAESLGGVESLISHAATMTHAGMSPQARAAAGISETLLRISTGIEDGEDLIADLGNGFRAANKG

SEQ ID NO: 34 - protein, 393 aa, Rhodobacter sphaeroides
  misc_feature 1..393: Rhodobacter sphaeroides ATCC 17029 (strain)
  metZ
MTKDWKTRTQLVHGGSRRSQYGEMAEAIFLTQGFVYDSAEQAEARFIETGADEFIYARYGNPTTRMFEERIAAVEGTEDAFATASGMAAIHGVLTSIVRAGDHLVAARALFGSCIYILEEVLGRFGVEVTFVDGTDLDQWRAAVRPGTKAVFFESVSNPTLEVADIGAIAEIAHAVGALVIVDNVFATPVFSTAVRQGADVVIYSATKHIDGQGRALGGVVCASQAFIRKVLEPFMKHTGGSMSPFNAWLMLNGMATLDLRCRAMADTAEKIARALEGHPQLGRVIHPALESHPQHDMAKAQMERPGTMIALDLAGGKEAAFRFLDALKIVKISNNLGDARSIATHPATTTHQRLSDAQKAHLGITPGLVRLSVGLEDADDLIADLKQALAVI

(19) United States
(12) Patent Application Publication
(10) Pub. No.: US 2024/0117327 A1
(43) Pub. Date: Apr. 11, 2024

(54) ENZYMATIC METHOD FOR PRODUCING L-GLUFOSINATE AND ITS PHOSPHOESTERS

(71) Applicant: Evonik Operations GmbH, Essen (DE)

(72) Inventors: Markus PÖTTER, Muenster (DE); Ludger Lautenschütz, Hanau (DE); Daniel Fischer, Hanau (DE); Jakob Müller, Haltern am See (DE)

(73) Assignee: Evonik Operations GmbH, Essen (DE)

(21) Appl. No.: 18/553,072

(22) PCT Filed: Mar. 28, 2022
(86) PCT No.: PCT/EP2022/058082

§ 371 Date: Sep. 28, 2023

(30) Foreign Application Priority Data
Apr. 1, 2021 (EP) 21166546.8

(51) Int. Cl. C12P 13/04; C12N 9/10

(52) U.S. Cl. CPC C12P 13/04; C12N 9/1085; C12Y 205/01048; C12Y 205/01049

(57) ABSTRACT

An enzymatically catalyzed method for producing L-glufosinate or a phosphoester of L-glufosinate can be performed. An activated L-homoserine H_{A} is reacted with a substrate S selected from methylphosphinic acid and the esters of methylphosphinic acid. The method makes accessible new substrates in the enzymatic production of L-glufosinate and its phosphoesters.

The present invention relates to an enzymatically catalyzed method for producing L-glufosinate (“L-GA” or “LGA”) or a phosphoester thereof. The method comprises a step in which an activated L-homoserine H_{A} is reacted with a substrate S selected from methylphosphinic acid and the esters of methylphosphinic acid. The invention makes accessible new substrates in the enzymatic production of L-glufosinate and its phosphoesters.

### 1. BACKGROUND OF THE INVENTION

Organic phosphor compounds, i.e. chemical agents comprising a carbon-phosphor bond, are widely applied as herbicides in the area of plant protection. Agents such as the herbicides glyphosate (Roundup®, Touchdown) and glufosinate (Basta®, Liberty®) as well as the growth regulator glyphosine (Polaris®) are used for this purpose (as described for example by G. Horlein, Rev. Environ. Contam. Toxicol. 1994, 138, 73-145).

The esters of P-methyl phosphinic acid (for example, P-methyl phosphinic acid butyl ester; “MPBE”; CAS-No: 6172-80-1) have a key role as synthetic building blocks in the synthesis of the non-selective herbicide glufosinate. These esters are accessible via two fundamental synthetic pathways (summarized in FIGS. 3 a and 3 b, p. 130, of the article of K. Haack, Chem. Unserer Zeit 2003, 37, 128-138):
a. Reacting diethyl chlorophosphite [CIP(OC₂H₅)₂] with CH₃MgCl provides methyl diethoxy phosphine [H₃CP(OC₂H₅)₂; “DEMP”; CAS-No. 15715-41-0], which is partially hydrolyzed to give the corresponding methylphosphinic acid ethyl ester (MPEE; CAS-Nr-16391-074).
b. Alternatively, methane can be reacted with phosphor trichloride at 500° C. to give methyl dichloro phosphane H₃CPCl₂. The latter can then be solvolyzed in alcohols to give the corresponding methyl phosphinic acid esters.

The esters of P-methyl phosphinic acid add to carbon-carbon double bonds regioselectively. This property is used in the synthesis of glufosinate for the formation of the second phosphor-carbon bond. For example, H₃CPH(O)OR (R=Alkyl) reacts with 1-cyano allyl acetate in an addition reaction to provide an intermediate. Subsequent exchange of the acetate substitent with ammonia and hydrolysis of the cyano group and the ester group of the phosphinic acid moiety give glufosinate.

Acrylic acid ester is a cheaper alternative starting material. It can react with the ester of P-methyl phosphinic acid to 3-[alkoxy(methyo)phosphinyl]propionic acid alkyl ester. Claisen reaction of this diesterwith diethy oxalate, hydrolysis and decarboxylation provide the corresponding α-keto acid, which can be reductively aminated to give glufosinate.

These and further synthetic mutes towards L-glufosinate are also described in the art, e.g. in WO 1999/009039 A1, EP 0 508 296 A1.

WO 2020/145513 A1 and WO 2020/145514 A1 describe a chemical route to L-glufosinate. In this route, a homoserine derivative such as 0-acetyl homoserine or O-succinyl homoserine is used as starting material and L-glufosinate is obtained by a sequence of reactions including lactonization and halogenation.

WO 2020/145627 A1 describes a similar route, wherein, during halogenation, a bromine derivative is obtained.

The route disclosed by CN 106083922 A is similar but starts off from L-methionine.

EP 2402453 A2 describes an enzymatic method for producing methionine by enzymatically reacting a mixture of methyl mercaptan and dimethyl sulfide with 0-acetyl homoserine or O-succinyl homoserine.

CN 108516991 A describes another synthetic pathway to L-glufosinate, starting with the azeotropic dehydration of L-homoserine to give L-3,6-bis(2-haloethyl)-2,5-diketopiperazine, followed by the introduction of a methylphosphinate diester group and hydrolysis.

A general disadvantage of all synthetic routes to glufosinate is that the obtained glufosinate is a racemic mixture. However, as there is no herbicidal activity of the D-enantiomer, L-glufosinate is the enantiomer of economical interest.

For the enantioselective syntheses of L-glufosinate, enzymatic pathways are described in the art.

WO 2017/151573 A1 discloses a two-step enzymatic synthesis of L-glufosinate from D-glufosinate.

In the first step, D-glufosinate is oxidatively deaminated to give 2-oxo-4-[hydroxy(methy)phosphinoyl]butyric acid (“PPO”), followed by the specific amination of PPO to L-glufosinate as the second step. The first step is carried out by the catalysis of a D-amino acid oxidase, the second step is catalyzed by a transaminase.

WO 2020/051188 A1 discloses a similar method of converting racemic glufosinate to the L-glufosinate enantiomer. In addition, it discloses a step in which the α-ketoacid or ketone byproduct formed during amination of PPO with an amine donor is converted by ketoglutarate decarboxylase to further shift the equilibrium to L-glufosinate.

WO 2019/018406 A1 discloses a method of purifying L-glufosinate from a mixture comprising L-glufosinate and glutamate. Glutamate is converted to pyroglutamate enzymatically by glutaminyl-peptidyl cyclotransferase, and L-glufosinate is then purified from the resulting mixture by ion-exchange.

The object of the present invention is to provide a further enzymatic process for producing L-gulfosinate in high enantiomeric excess. In particular, such process should allow to use new substrates which heretofore were not used in the enzymatic synthesis of L-glufosinate.

### 2. SUMMARY OF THE INVENTION

The present invention solves the problems mentioned above by providing a method for producing L-glufosinate from a substrate that has not been used in the enzymatic production of L-glufosinate before. In particular, the present invention provides a method for producing L-glufosinate or a phosphoester of L-glufosinate from methylphosphinic acid and its esters using an enzymatically catalyzed pathway. These phosphor compounds thus serve as alternative substrates in the production of L-glufosinate, allowing for flexibility of production where there is no reliance on the known substrates that are currently being used for L-glufosinate production.

In particular, this object is achieved by the present invention which relates to an enzymatically catalyzed method for producing L-glufosinate or a phosphoester thereof, comprising a step (a) in which an activated L-homoserine H_{A} is reacted with a substrate S to produce these compounds.

### 3. BRIEF DESCRIPTION OF THE DRAWINGS

FIG. 1 shows the pET-26b(+)_metY-Cg plasmid map.

FIG. 2 shows the pET-26b(+)_metY_P2T plasmid map.

FIG. 3 shows the pET-26b(+)_metZ-Cv plasmid map.

FIG. 4 shows the pET-26b(+)_metZ-Hn plasmid map.

### 4. DETAILED DESCRIPTION OF THE INVENTION

It was surprisingly found that certain phosphor-containing compounds, namely methylphosphinic acid and esters of methylphosphinic acid, can react under enzymatic catalysis with activated L-homoserine, thus opening a new synthetic pathway to L-glufosinate and L-glufosinate posphoesters. This was especially surprising because similar compounds such as DEMP did not react in the analogous reaction with activated L-homoserine.

The present invention thus relates to an enzymatically catalyzed method for producing L-glufosinate or a phosphoester thereof, comprising a step (a) in which an activated L-homoserine H_{A} is reacted with a substrate S of the following structure (I) to produce a compound of the following structure (III),

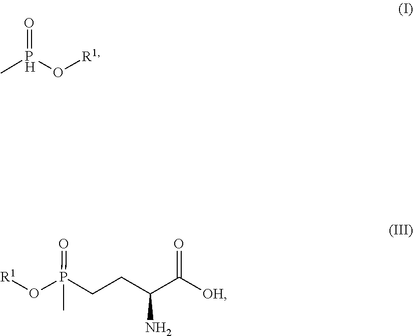

wherein R¹ is selected from hydrogen, alkyl, alkenyl, alkinyl, hydroxyalkyl, aryl.

The compounds denoted as “L-glufosinate or a phosphoester thereof” according to the invention are represented by structure (III). When R¹=hydrogen in structure (III), the compound is L-GA.

When R¹ is selected from alkyl, alkenyl, alkinyl, hydroxyalkyl, aryl in structure (III), the compound is a phosphoester of L-GA.

The activated L-homoserine H_{A} has the following structure (II):

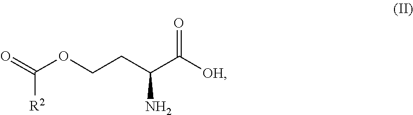

wherein R² is a hydrocarbon group with 1 to 15 carbon atoms which optionally comprises at least one functional group selected from OH, COOH, NH.

The reaction in step (a) is catalyzed by at least one enzyme selected from the group consisting of a sulfhydrylase E₁, a cystathionine γ-synthase E₂.

### 4.1 Substrate S

The substrate S according to the invention is selected from the group consisting of methylphosphinic acid and the esters of methylphosphinic acid.

The substrate S has the structure (I). In structure (I), R¹ is selected from hydrogen, alkyl, alkenyl, alkinyl, hydroxyalkyl, aryl,
preferably selected from hydrogen, alkyl,
preferably selected from hydrogen, alkyl with 1 to 6, preferably 1 to 4 carbon atoms,
more preferably selected from hydrogen, methyl, ethyl, iso-propyl, n-propyl, n-butyl, seoc-butyl, iso-butyl, ter-butyl, even more preferably selected from hydrogen, methyl, ethyl, n-butyl, even more preferably selected from hydrogen, methyl, n-butyl, preferably selected methyl, n-butyl. Most preferably, R¹ is n-butyl.

When R¹=hydrogen in structure (I), the compound is methylphosphinic acid.

When R¹ is selected from alkyl, alkenyl, alkinyl, hydroxyalkyl, aryl in structure (I), the compound is an ester of methylphosphinic acid.

R¹ in structure (I) and structure (III) is the same.

### 4.2 Activated L-Homoserine H_{A}

The other reaction partner in the reaction according to the present invention is activated L-homoserine H_{A}.

The skilled person is aware of activated L-homoserine H_{A} (sometimes also denoted as “L-methionine precursor”, e.g. in WO 2008/013432 A1), which in particular means O-acyl L-homoserine.

### 4.2.1 Activated L-Homoserine H_{A}

The activated L-homoserine has a chemical structure (II) as follows:

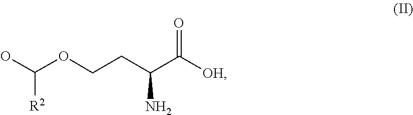

wherein R² is a hydrocarbon group with 1 to 15 carbon atoms which optionally comprises at least one functional group selected from OH, COOH, NH.

More preferably, the activated L-homoserine is selected from the group consisting of O-acetyl-L-homoserine [structure (II-A)], O-succinyl-L-homoserine [structure (II-B)], O-propionyl-L-homoserine [structure (II-C)], O-acetoacetyl-L-homoserine [structure (II-D)], O-coumaroyl-L-homoserine [structure (II-E)], O-malonyl-L-homoserine [structure (II-F)], O-hydroxymethylglutaryl-L-homoserine [structure (II-G)], and O-pimelyl-L-homoserine [structure (II-H)].

Even more preferably, the activated L-homoserines is selected from the group consisting of O-acetyl-L-homoserine [structure (II-A)], O-succinyl-L-homoserine [structure (II-B)].

Most preferably, the activated L-homoserine is O-acetyl-L-homoserine [structure (II-A)].

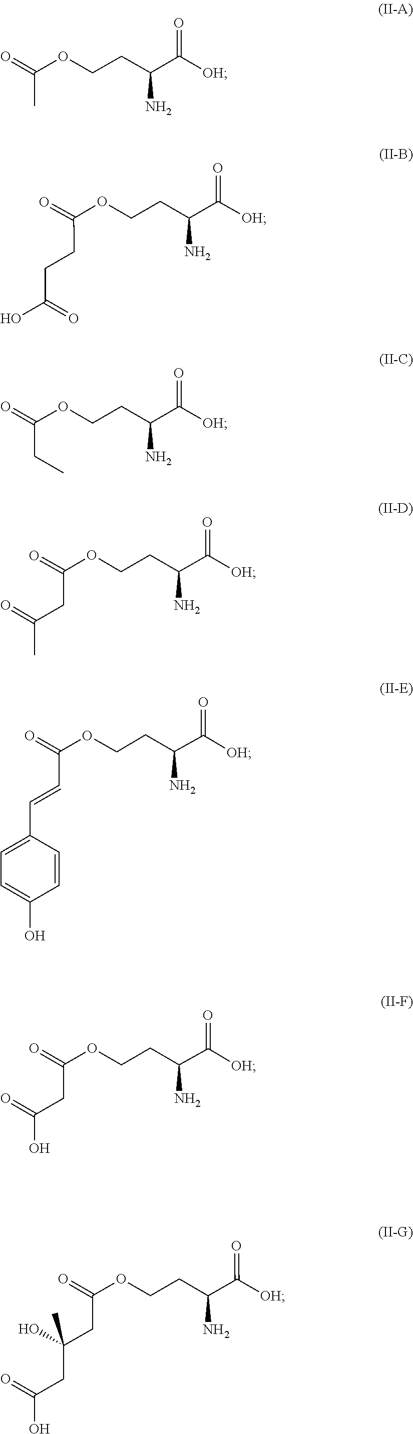

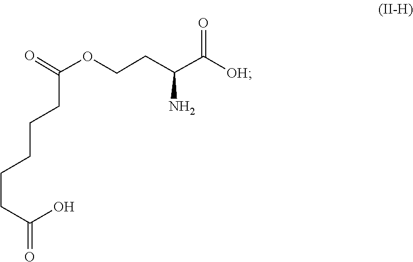 [SMILES: N[C@@H](CCOC(=O)CCCCCC(=O)O)C(=O)O]

### 4.2.2 Chemical Synthesis of Activated L-Homoserine H_{A}

The activated L-homoserine H_{A} used in the method of the present invention can be obtained by organochemical synthesis mutes known to the skilled person. For example, the synthesis of O-succinyl homoserine is described in M. Flavin, C. Slaughter, Biochemistry 1965, 4, 1370-1375. The synthesis of O-acetyl-homoserine is described by S. Nagai, M. Flavin, Methods in Enzymology, Metabolism of Amino Acids and Amines Part B 1971, 17(Part B), 423-424.

The chemical synthesis of the potential precursor is described for example by M. D. Armstrong, J. Am. Chem. Soc. 1948, 70, 1756-1759.

### 4.2.3 Biotechnological Synthesis of Activated L-Homoserine H_{A}

Alternatively, and preferably, the activated L-homoserine H_{A} used in the present invention is obtained by biotechnological means. For example, this is described in WO 2008/013432 A1 or by H. Kase, K. Nakayama, Agr. Biol. Chem. 1974, 38, 2021-2030.

The strain producing activated L-homoserine H_{A} is preferably selected from the group consisting of Escherichia sp., Erwinia sp., Serratia sp., Providencia sp., Corynebacterum sp., Pseudomonas sp., Leptospira sp., Salmonella sp., Brevibacterium sp., Hypomononas sp., Chromobacterium sp., Norcardia sp., fungi, which are in particular yeasts.

Biotechnological processes for obtaining L-homoserine are also described in the art, e.g. in U.S. Pat. Nos. 3,598,701, 6,303,348 B1, EP 0 994 190 A2, EP 1 149 911 A2, WO 2004/067757 A1.

### 4.3 Enzyme

The method according to the present invention is enzymatically catalyzed.

The term “enzyme” means any substance composed wholly or largely of protein or polypeptides that catalyzes or promotes, more or less specifically, one or more chemical or biochemical reactions.

Any of the enzymes used according to any aspect of the present invention may be an isolated enzyme. In particular, the enzymes used according to any aspect of the present invention may be used in an active state and in the presence of all cofactors, substrates, auxiliary and/or activating polypeptides or factors essential for its activity.

In particular, this also means that the terms “cystathionine γ-synthase” and “sulfhydrylase”, in particular “O-acetyl homoserine sulfhydrylase” or “O-succinyl homoserine sulfhydrylase” comprise the respective enzymes in combination with all the cofactors necessary for their function. In particular, this cofactor is pyridoxal 5-phosphate mono-hydrate (“PMP”).

A “polypeptide” is a chain of chemical building blocks called amino acids that are linked together by chemical bonds called peptide bonds. A protein or polypeptide, including an enzyme, may be “native” or “wild-type”, meaning that it occurs in nature or has the amino acid sequence of a native protein, respectively. These terms are sometimes used interchangeably. A polypeptide may or may not be glycosylated.

The enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule. As a further example, a polypeptide is recombinant if it is identical to a polypeptide sequence occurring in nature but has been engineered to contain one or more point mutations that distinguish it from any polypeptide sequence occurring in nature.

The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell.

The term “isolated”, as used herein, means that the enzyme of interest is enriched compared to the cell in which it occurs naturally. The enzyme may be enriched by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

### 4.3.1 Enzymes E₁ and E₂

Step (a) of the method according to the invention is catalysed by at least one enzyme selected from the group consisting of a sulfhydrylase E₁ and a cystathionine γ-synthase E₂.

A sulfhydrylase is known to the skilled person as an enzyme that catalyzes at least one of the following reactions <1A>, <1B>:

O-acetyl-L-homoserine+methanethiol→L-methionine+acetate. <1A>:

O-succinyl-L-homoserine+methanethiol→L-methionine+succinate. <1B>:

A sulfhydrylase that has a higher catalytic activity for reaction <1A> than reaction <1B> may be denoted as an “O-acetyl-L-homoserine sulfhydrylase”.

A sulfhydrylase that has a higher catalytic activity for reaction <1B> than reaction <1A> may be denoted as an “O-succinyl-L-homoserine sulfhydrylase”.

A cystathionine γ-synthase is known to the skilled person as an enzyme that catalyzes at least the following reaction <2>:

O-succinyl-L-homoserine+L-cysteine→L-cystathionine+succinate <2>:

In a preferred embodiment, step (a) of the method according to the invention is catalysed by a sulfhydrylase E₁, which is even more preferably an O-acetyl homoserine suifhydrylase or an O-succinyl homoserine sulfhydrylase, most preferably an O-acetyl homoserine sulfhydrylase.

The sulfhydrylase or the cystathionine γ-synthase that may be used in step (a) of the method according to the invention may be derived from Bacillus sp., in particular Bacillus subtilis; Bradyrhizobium sp., in particular Bradyrhizobium japonicum; Brevibacterium sp.; Colwellia sp., in particular Colwellia psycherythraea; Corynebacterium sp., in particular Corynebacterium glutamicum, Corynebacterium humireducens; Chromobacterium sp., in particular Chromobacterium violaceum; Erwinia sp.; Escherichia sp., in particular Escherichia coli; Hyphomonas sp., in particular Hyphomonas neptunium; Klebsiella sp., in particular Klebsiella pneumoniae; Leptospira sp., in particular Leptospira interrogans; Methylobacillus sp., in particular Methylobacillus flagellates; Methylococcus sp., in particular Methylococcus capsulatus; Nitrosomonas sp., in particular Nitrosomonas europaea; Nocardia sp., in particular Nocardia farcinica; Providencia sp.; Pseudomonas sp., in particular Pseudomonas aeruginosa; Rhodobacter sp., in particular Rhodobacter sphaeroides; Salmonella sp., in particular Salmonella enterica; Serratia sp.; Shigella sp., in particular Shigella flexneri; fungi, which are preferably yeast, more preferably Saccharomyces sp., even more preferably Saccharomyces cerevisiae.

The sulfhydrylase enzyme that may be used in the method according to the present invention may be an O-acetyl-L-homoserine sulfhydrylase categorized in the EC class EC 2.5.1.49 or an O-succinyl-L-homoserine suifhydrylase categorized in the EC class EC 2.5.1.-.

These enzymes are part of the direct sulfurylation pathway for methionine biosynthesis and PMP-dependent. They are described e.g. by M. P. Feria and W. M. Patrick, Microbiology 2014, 160, 1571-1584.

WO 02/18613 A1, WO 2007/024933 A2, EP 2 657 345 A1, EP 2 657 250 A2, WO 2015/165746 A1 and WO 2008/013432 A1 disclose examples of enzymes having O-acetyl-L-homoserine sulfhydrylase and O-succinyl-L-homoserine sulfhydrylase activity according to the invention.

An O-acetyl-L-homoserine sulfhydrylase suitable for the method according to the present invention may originate from Corynebacterium sp., in particular Corynebacterium glutamicum or Corynebacterium humireducens; Leptospira sp., in particular Leptospira interrogans; Pseudomonas sp., in particular Pseudomonas aeruginosa; yeast, in particular Saccharomyces sp., preferably Saccharomyces cerevisiae. A preferable O-acetyl-L-homoserine sulfhydrylase suitable for the method according to the present invention may originate from Corynebacterium sp., more preferably Corynebacterium glutamicum or Corynebacterium humireducens, even more preferably Corynebacterium glutamicum, even more preferably Corynebacterium glutamicum ATCC 13032.

An O-succinyl-L-homoserine sulfhydrylase suitable for the method according to the present invention may originate from Bradyrhizobium sp., in particular Bradyrhizobium japonicum; Chromobacterium sp., in particular Chromobacterium violaceum; Hyphomonas sp., in particular Hyphomonas neptunium; Methylobacillus sp., in particular Methylobacillus flagellatus; Methylococcus sp., in particular Methylococcus capsulatus; Nitrosomas sp., in particular Nitrosomas europaea; Nocardia sp., Nocardia farcinica; Pseudomonas sp., in particular Pseudomonas aeruginosa, Pseudomonas putida; Rhodobacter sp., in particular Rhodobacter sphaeroides.

A preferable O-succinyl-L-homoserine sulfhydrylase suitable for the method according to the present invention may originate from Chromobacterium sp., in particular Chromobacterium violaceum; Hyphomonas sp., in particular Hyphomonas neptunium. Even more preferably, it may originate from Chromobacterium violaceum ATCC 12472, Hyphomonas neptunium ATCC 15444.

The cystathionine γ-synthase that may be used in the method according to the present invention may be a cystathionine γ-synthase categorized in the EC class EC 2.5.1.48.

These enzymes are PMP-dependent and are described e.g. by T. Clausen, R. Huber, L. Prade, M. C. Wahl and A. Messerschmidt, The EMBO Journal 1998, 17, 6827-8838.

A cystathionine γ-synthase suitable for the method according to the present invention may originate from Escherichia coli, in particular Escherichia coli; Corynebacterium sp., in particular Corynebacterium glutamicum; Klebsiella sp., in particular Klebsiella pneumoniae; Bacillus sp., in particular Bacillus subtilis; Shigella sp., in particular Shigella flexneri; Colwellia sp., in particular Colwellia psychrerythraea; Salmonella sp., in particular Salmonella enterica.

The respective sequences can be derived from databases such as the Braunschweig Enzyme Database (BRENDA, Germany, available underwww.brenda-enzymes.org/index.php), the National Center for Biotechnological Information (NCBI, available under https://www.ncbi.nlm.nih.gov/) or the Kyoto Encyclopedia of Genes and Genomes (KEGG, Japan, available under www.https://www.genome.jp/kegg/).

The following table 1 gives preferred examples for sulfhydrylases and cystathionine γ-synthases that may be used in step (a) of the method according to the invention. The genes encoding sulfhydrylase are indicated as “metY”, “met17”, and “Met17” for O-acetyl-L-homoserine sulfhydrylase (“AHS”) and “metZ” for O-succinyl-L-homoserine sulfhydrylase (“SHS”). The gene encoding cystathionine γ-synthase (“CGS”) is indicated as “metB” and “met”.

TABLE 1

| | | | NCBI | SEQ ID NO: of |
|---|---|---|---|---|
| Strain | Gene name | Gene ID/locus tag | accession | the polypeptide |
| Corynebacterium glutamicum ATCC 13032 | metY | NCgl0625 | NC_003450 | SEQ ID NO: 2 |
| Corynebacterium glutamicum ATCC 13032 | metY_P2T | — | — | SEQ ID NO: 6 |
| Chromobacterium violaceum ATCC 12472 | metZ | CV_2725 | AE016825 | SEQ ID NO: 10 |
| Hyphomonas neptunium ATCC 15444 | metZ | HNE_2672 | CP000158 | SEQ ID NO: 14 |
| Escherichia coli K12 MG1655 | metB | b3939 | U00096 | SEQ ID NO: 17 |
| Pseudomonas aeruginosa PAO1 | metZ | 880476/PA3107 | AE004091 | SEQ ID NO: 18 |
| Pseudomonas aeruginosa PAO1 | metY | 881205/PA5025 | AE004091 | SEQ ID NO: 19 |
| Pseudomonas putida KT2440 | metZ | PP_RS10380 | NC_002947 | SEQ ID NO: 20 |
| Corynebacterium glutamicum ATCC 13032 | metB | NCgl2360/CGL_RS12110 | NC_003450 | SEQ ID NO: 21 |
| Leptospira interrogans serovar Lai str. 56601 | met17 | LA_2062 | AE010300 | SEQ ID NO: 22 |
| Saccharomyces cerevisiae S288C | Met17 | Met17 | NC_001144 | SEQ ID NO: 23 |
| Nocardia farcinica IFM 10152 | metZ | NFA_53590 | AP006618 | SEQ ID NO: 24 |
| Bradyrhizobium japonicum USDA 6 | metZ | BJ6T_RS05520 | NC_017249 | SEQ ID NO: 25 |
| Methylococcus capsulatus str. Bath | metZ | MCA2488 | AE017282 | SEQ ID NO: 26 |
| Methylobacillus flagellatus KT | “metZ” | MFLA_RS08660 | NC_007947 | SEQ ID NO: 27 |
| Nitrosomonas europaea ATCC 19718 | metZ | ALW85_RS03655 | NC_004757 | SEQ ID NO: 28 |
| Klebsiella pneumoniae subsp. pneumoniae MGH 78578 | metB | KPN_RS22820 | NC_009648 | SEQ ID NO: 29 |
| Bacillus subtilis subsp. subtilis str. 168 | metI | BSU_11870 | NC_000964 | SEQ ID NO: 30 |
| Shigella flexneri 2a 2457T ATCC 700930 | metB | S3730 | AE014073 | SEQ ID NO: 31 |
| Colwellia psychrerythraea 34H ATCC BAA-681 | metB | CPS_0455 | CP000083 | SEQ ID NO: 32 |
| Salmonella enterica serovar Paratyphi A ATCC 9150 | metB | SPA3943 | CP000026 | SEQ ID NO: 33 |
| Rhodobacter sphaeroides ATCC 17029 | metZ | RSPH17029_RS02535 | NC_009049 | SEQ ID NO: 34 |

In a preferred embodiment of the method of the present invention, the reaction in step (a) is catalyzed by at least one enzyme selected from the group consisting of a sulfhydrylase E₁, a cystathionine γ-synthase E₂, wherein the polypeptide sequence of the sulfhydrylase enzyme E₁ is selected from the group consisting of:
O-acetyl homoserine sulfhydrylases selected from the group consisting of SEQ ID NO: 2 and variants of SEQ ID NO: 2, SEQ ID NO: 6 and variants of SEQ ID NO: 6, SEQ ID NO: 19 and variants of SEQ ID NO: 19, SEQ ID NO: 22 and variants of SEQ ID NO: 22, SEQ ID NO: 23 and variants of SEQ ID NO: 23, and
O-succinyl homoserine sulfhydrylases selected from the group consisting of SEQ ID NO: 10 and variants of SEQ ID NO: 10, SEQ ID NO: 14 and variants of SEQ ID NO: 14, SEQ ID NO: 18 and variants of SEQ ID NO: 18, SEQ ID NO: 20 and variants of SEQ ID NO: 20, SEQ ID NO: 24 and variants of SEQ ID NO: 24, SEQ ID NO: 25 and variants of SEQ ID NO: 25, SEQ ID NO: 26 and variants of SEQ ID NO: 26, SEQ ID NO: 27 and variants of SEQ ID NO: 27, SEQ ID NO: 28 and variants of SEQ ID NO: 28, SEQ ID NO: 34 and variants of SEQ ID NO: 34,
and wherein the polypeptide sequence of the cystathionine γ-synthase E₂ is selected from the group consisting of SEQ ID NO: 17 and variants thereof, SEQ ID NO: 21 and variants thereof, SEQ ID NO: 29 and variants thereof, SEQ ID NO: 30 and variants thereof, SEQ ID NO: 31 and variants thereof, SEQ ID NO: 32 and variants thereof, SEQ ID NO: 33 and variants thereof.

In a more preferred embodiment of the method of the present invention, the reaction in step (a) is catalyzed by a sulfhydrylase E₁ selected from the group consisting of
O-acetyl homoserine sulfhydrylases selected from the group consisting of SEQ ID NO: 2 and variants of SEQ ID NO: 2, SEQ ID NO: 6 and variants of SEQ ID NO: 6, SEQ ID NO: 19 and variants of SEQ ID NO: 19, SEQ ID NO: 22 and variants of SEQ ID NO: 22, SEQ ID NO: 23 and variants of SEQ ID NO: 23, and
O-succinyl homoserine sulfhydrylases selected from the group consisting of SEQ ID NO: 10 and variants of SEQ ID NO: 10, SEQ ID NO: 14 and variants of SEQ ID NO: 14, SEQ ID NO: 18 and variants of SEQ ID NO: 18, SEQ ID NO: 20 and variants of SEQ ID NO: 20, SEQ ID NO: 24 and variants of SEQ ID NO: 24, SEQ ID NO: 25 and variants of SEQ ID NO: 25, SEQ ID NO: 26 and variants of SEQ ID NO: 26, SEQ ID NO: 27 and variants of SEQ ID NO: 27, SEQ ID NO: 28 and variants of SEQ ID NO: 28, SEQ ID NO: 34 and variants of SEQ ID NO: 34.

In an even more preferred embodiment of the method of the present invention, the reaction in step (a) is catalyzed by a sulfhydrylase E₁ selected from the group consisting of O-acetyl homoserine sulfhydrylases selected from the group consisting of SEQ ID NO: 2 and variants of SEQ ID NO: 2, SEQ ID NO: 6 and variants of SEQ ID NO: 6 and O-succinyl homoserine sulfhydrylases selected from the group consisting of SEQ ID NO: 10 and variants of SEQ ID NO: 10, SEQ ID NO: 14 and variants of SEQ ID NO: 14.

In an even more preferred embodiment of the method of the present invention, the reaction in step (a) is catalyzed by a sulfhydrylase E₁ selected from the group consisting of O-acetyl homoserine sulfhydrylases selected from the group consisting of SEQ ID NO: 2, SEQ ID NO: 6, and O-succinyl homoserine sulfhydrylases selected from the group consisting of SEQ ID NO: 10, SEQ ID NO: 14.

The term “variant” is further explained below (item 4.3.3.1). In the context of the present application, it is understood to mean a polypeptide sequences with at least 80% sequence identity to the respective polypeptide sequence.

### 4.3.2 Methods for Obtaining Enzymes

The enzymes that can be used in the method according to the present invention can be synthesized by methods that are known to the skilled person.

One approach is to express the enzyme(s) in microorganism(s) such as Escherichia coli (=“E. coli”), Saccharomyces cerevisiae, Pichia pastoris, and others, and to add the whole cells to the reactions as whole cell biocatalysts. Another approach is to express the enzyme(s), lyse the microorganisms, and add the cell lysate. Yet another approach is to purify, or partially purify, the enzyme(s) from a lysate and add pure or partially pure enzyme(s) to the reaction. If multiple enzymes are required for a reaction, the enzymes can be expressed in one or several microorganisms, including expressing all enzymes within a single microorganism.

For example, the skilled person can obtain the enzymes according to the invention by expression, in particular, overexpression, (hereinafter, “expression, in particular overexpression” is abbreviated as (over)expression”, and “express, in particular overexpress” is abbreviated as “(over)express”) of these enzymes in a cell and subsequent isolation thereof, e.g. as described in DE 100 31 999 A1. Episomal plasmids, for example, are employed for increasing the expression of the respective genes. In such plasmids, the nucleic acid molecule to be (over)expressed or encoding the polypeptide or enzyme to be (over)expressed may be placed under the control of a strong inducible promoter such as the lac promoter, located upstream of the gene. A promoter is a DNA sequence consisting of about 40 to 50 base pairs which constitutes the binding site for an RNA polymerase holoenzyme and the transcriptional start point (M. Pátek, J. Holátko, T. Busche, J. Kalinowski, J. Nešvera, Microbial Biotechnology 2013, 6, 103-117), whereby the strength of expression of the controlled polynucleotide or gene can be influenced. A “functional linkage” is obtained by the sequential arrangement of a promoter with a gene, which leads to a transcription of the gene.

Suitable strong promoters or methods of producing such promoters for increasing expression are known from the literature (e.g. S. Lisser & H. Margalit, Nucleic Acid Research 1993, 21, 1507-1516; M. Paték and J. Nesvera in H. Yukawa and M Inui (eds.), Corynebacterium glutamicum, Microbiology Monographs 23, Springer Verlag Berlin Heidelberg 2013, 51-88; B. J. Eikmanns, E. Kleinertz, W. Liebl, H. Sahm, Gene 1991, 102, 93-98). For instance, native promoters may be optimized by altering the promoter sequence in the direction of known consensus sequences with respect to increasing the expression of the genes functionally linked to these promoters (M. Pitek, B. J. Eikmanns, J. Pitek, H. Sahm, Microbiology 1996, 142, 1297-1309; M. Pitek, J. Holitko, T. Busche, J. Kalinowski, J. Nešvera, Microbial Biotechnology 2013, 6, 103-117).

Constitutive promoters are also suitable for the (over)expression, in which the gene encoding the enzyme activity is expressed continuously under the control of the promoter such as, for example, the glucose dependent deo promoter. Chemically induced promoters are also suitable, such as tac, lac or rp. The most widespread system for the induction of promoters is the lac operon of E. coli. In this case, either lactose or isopropyl ß-D-thiogalactopyranoside (IPTG) is used as inducer. Also, systems using arabinose (e.g. the pBAD system) or rhamnose (e.g. E. coli KRX) are common as inducers. A system for physical induction is, for example, the temperature-induced cold shock promoter system based on the E. coli cspA promoter from Takara or Lambda PL and also osmotically inducible promoters, for example, osmB (e.g. WO 95/25785 A1).

Suitable plasmids or vectors are in principle all embodiments available for this purpose to the person skilled in the art. The state of the art describes standard plasmids that may be used for this purpose, for example the pET system of vectors exemplified by pET-3a or pET-26b(+) (commercially available from Novagen). Further plasmids and vectors can be taken, for example, from the brochures of the companies Novagen, Promega, New England Biolabs, Clontech or Gibco BRL. Further preferred plasmids and vectors can be found in: Glover, D. M. (1985) DNA cloning: a practical approach, Vol. I-III, IRL Press Ltd., Oxford; Rodriguez, R. L. and Denhardt, D. T (eds) (1988) Vectors: a survey of molecular cloning vectors and their uses, 179-204, Butterworth, Stoneham; Goeddel, D. V. (1990) Systems for heterologous gene expression, Methods Enzymol. 185, 3-7; Sambrook, J.; Fritsch, E. F. and Maniatis, T. (1989), Molecular cloning: a laboratory manual, 2nd ed., Cold Spring Harbor Laboratory Press, New York.

The plasmid vector, which contains the gene to be amplified, is then converted to the desired strain, e.g. by conjugation or transformation. The method of conjugation is described, for example, by A. Schäfer, J. Kalinowski, A. Pühler, Applied and Environmental Microbiology 1994, 60, 756-759. Methods for transformation are described, for example, in G. Thierbach, A. Schwarzer, A. Pühler, Applied Microbiology and Biotechnology 1988, 29, 356-362, L. K. Dunican & E. Shivnan, Bio/Technology 1989, 7, 1067-1070 and A. Tauch, O. Kirchner, L. Wehmeier, J. Kalinowski, A. Pühler, FEMS Microbiology Letters 1994, 123, 343-347. After homologous recombination by means of a “cross-over” event, the resulting strain contains at least two copies of the gene concerned.

The desired enzyme can be isolated by disrupting cells which contain the desired activity in a manner known to the person skilled in the art, for example with the aid of a ball mill, a French press or of an ultrasonic disintegrator and subsequently separating off cells, cell debris and disruption aids, such as, for example, glass beads, by centrifugation for 10 minutes at 13000 rpm and 4° C. Using the resulting cell-free crude extract, enzyme assays with subsequent LC-ESI-MS detection of the products can then be carried out. Alternatively, the enzyme can be enriched in the manner known to the person skilled in the art by chromatographic methods (such as nickel-nitrilotriacetic acid affinity chromatography, streptavidin affinity chromatography, gel filtration chromatography or ion-exchange chromatography) or else purified to homogeneity.

Whether or not a nucleic acid or polypeptide is (over)expressed, may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions.

### 4.3.3 Definitions

4.3.3.1 “Variants”

In the context of the present invention, the term “variant” with respect to polypeptide sequences refers to a polypeptide sequence with a degree of identity to the reference sequence of at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%. In still further particular embodiments, the degree of identity is at least 98.0%, more preferably at least 98.2%, more preferably at least 98.4%, more preferably at least 98.6%, more preferably at least 98.8%, more preferably at least 99.0%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, or at least more preferably at least 99.9%. It goes without saying that a “variant” of a certain polypeptide sequence is not identical to the polypeptide sequence.

Such variants may be prepared by introducing deletions, insertions, substitutions, or combinations thereof, in particular in amino acid sequences, as well as fusions comprising such macromolecules or variants thereof.

Modifications of amino acid residues of a given polypeptide sequence which lead to no significant modifications of the properties and function of the given polypeptide are known to those skilled in the art. Thus for example many amino acids can often be exchanged for one another without problems; examples of such suitable amino acid substitutions are: Ala by Ser; Arg by Lys; Asn by Gin or His; Asp by Glu; Cys by Ser; Gin by Asn; Glu by Asp; Gly by Pro; His by Asn or Gin; lie by Leu or Val; Leu by Met or Val; Lys by Arg or Gin or Glu; Met by Leu or Ile; Phe by Met or Leu or Tyr; Ser by Thr; Thr by Ser; Trp by Tyr; Tyr by Trp or Phe; Val by Ile or Leu. It is also known that modifications, particularly at the N- or C-terminus of a polypeptide in the form of for example amino acid insertions or deletions, often exert no significant influence on the function of the polypeptide.

In line with this, preferable variants according to the invention of any of SEQ ID NO: 2, SEQ ID NO: 6, SEQ ID NO: 19, SEQ ID NO: 22, SEQ ID NO: 23, SEQ ID NO: 10, SEQ ID NO: 14, SEQ ID NO: 18, SEQ ID NO: 20, SEQ ID NO: 24, SEQ ID NO: 25, SEQ ID NO: 26, SEQ ID NO: 27, SEQ ID NO: 28, SEQ ID NO: 34, SEQ ID NO: 17, SEQ ID NO: 21, SEQ ID NO: 29, SEQ ID NO: 30, SEQ ID NO: 31, SEQ ID NO: 32, and SEQ ID NO: 33, respectively, have a polypeptide sequence that comprises the amino acids of the respective sequence that are essential for the function, for example the catalytic activity of a protein, or the fold or structure of the protein. The other amino acids may be deleted, substituted or replaced by insertions or essential amino acids are replaced in a conservative manner to the effect that the activity of the enzyme, in particular the sulfhydrylase or the cystathionine γ-synthase, is preserved.

4.3.3.2 “Sequence Identity”

The person skilled in the art is aware that various computer programs are available for the calculation of similarity or identity between two nucleotide or amino acid sequences.

Preferred methods for determining the identity initially generate the greatest alignment between the sequences to be compared. Computer programs for determining the identity include, but are not limited to, the GCG program package including
GAP [J. Deveroy et al., Nucleic Acid Research 1984, 12, page 387, Genetics Computer Group University of Wisconsin, Medicine (WI)], and
BLASTP, BLASTN and FASTA (S. Altschul et al., Journal of Molecular Biology 1990, 215, 403-410). The BLAST program can be obtained from the National Center for Biotechnology Information (NCBI) and from other sources (BLAST Handbook, S. Altschul et al., NCBI NLM NIH Bethesda ND 22894; S. Altschul et al., above).

For instance, the percentage identity between two amino acid sequences can be determined by the algorithm developed by S. B. Needleman and C. D. Wunsch, J. Mol. Biol. 1970, 48, 443-453, which has been integrated into the GAP program in the GCG software package, using either a BLOSUM62 matrix or a PAM250 matrix, a gap weight of 16, 14, 12, 10, 8, 6 or 4 and a length weight of 1, 2, 3, 4, 5 or 6. The person skilled in the art will recognize that the use of different parameters will lead to slightly different results, but that the percentage identity between two amino acid sequences overall will not be significantly different. The BLOSUM62 matrix is typically used applying the default settings (gap weight: 12, length weight: 1).

In the context of the present invention, a sequence identity of 80% according to the above algorithm means 80% homology. The same applies to higher identities.

Most preferably, the degree of identity between sequences is determined in the context of the present invention by the programme “Needle” using the substitution matrix BLOSUM62, the gap opening penalty of 10, and the gap extension penalty of 0.5. The Needle program implements the global alignment algorithm described in S. B. Needleman and C. D. Wunsch, J. Mol. Biol. 1970, 48, 443-453. The substitution matrix used according to the present invention is BLOSUM62, gap opening penalty is 10, and gap extension penalty is 0.5. The preferred version used in the context of this invention is the one presented by F. Madeira, Y. M. Park, J. Lee, N. Buso, T. Gur, N. Madhusoodanan, P. Basutkar, A. R. N. Tivey, S. C. Potter, R. D. Finn, Nucleic Acids Research 2019, 47, W836-W841, Web Server issue (preferred version accessible online on Mar. 31, 2021 via https://www.ebi.ac.uk/Tools/psa/emboss_needle/).

In a particular embodiment, the percentage of identity of an amino acid sequence of a polypeptide with, or to, a reference polypeptide sequence is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the longest of the two amino acid sequences, and iv) converting the result of the division of iii) into percentage.

4.3.3.3 “Preferred Assay to Identify Particularly Active Variants”

4.3.3.3.1 Assay A

Especially preferable polypeptide variants of any of SEQ ID NO: 2, SEQ ID NO: 6, SEQ ID NO: 19, SEQ ID NO: 22, SEQ ID NO: 23, SEQ ID NO: 10, SEQ ID NO: 14, SEQ ID NO: 18, SEQ ID NO: 20, SEQ ID NO: 24, SEQ ID NO: 25, SEQ ID NO: 26, SEQ ID NO: 27, SEQ ID NO: 28, SEQ ID NO: 34, SEQ ID NO: 17, SEQ ID NO: 21, SEQ ID NO: 29, SEQ ID NO: 30, SEQ ID NO: 31, SEQ ID NO: 32, and SEQ ID NO: 33, respectively, in the context of the present invention may be identified by the skilled person as those displaying activity in the following assay (“Assay A”).

Assay A is carried out by the following steps:
A1) First, the activity of the variant to be tested is determined by the following steps A1.1), A1.2), and A1.3) as follows:
A1.1) 990 μl of a reaction solution containing phosphate buffer (0.1 M, pH 7.5), 3 mM O-acetyl L-homoserine-HCl, 10 μM pyridoxal 5-phosphate mono-hydrate and 1.0 nmol of the polypeptide to be tested are prepared and heated to 50° C.
A1.2) The reaction is started by adding 10 μl of a 200 mM solution of butyl-P-methylphosphinate (MPBE, CAS #6172-80-1). The concentration of MPBE in the reaction solution is 2 mM.
A1.3) After addition of the MPBE, the reaction is conducted for 120 minutes at 50° C. Then, the reaction is stopped by adding 10 μl 1% formate solution and cooling on ice.
A2) Then, a blank test is carried out by the following steps A2.1), A2.2), and A2.3) as follows: A2.1) 990 μl of a reaction solution containing phosphate buffer (0.1 M, pH 7.5), 3 mM O-acetyl L-homoserine-HCl, 10 μM pyridoxal 5-phosphate mono-hydrate are prepared and heated to 50° C.
A2.2) The reaction is started by adding 10 μl of a 200 mM solution of butyl-P-methylphosphinate (MPBE, CAS #6172-80-1).
A2.3) After addition of the MPBE, the reaction is conducted for 120 minutes at 50° C. Then, the reaction is stopped by adding 10 μl 1% formate solution and cooling on ice.
A3) Finally, the amount (in mole) of the butyl-phosphoester of L-GA (i.e. the compound according to formula (III) with R¹=n-butyl) obtained in the reaction solution in A1.3) and A2.3), respectively, is determined and compared, preferably by LC-MS analysis described under item 5.7.
A4) If the amount of butyl-phosphoester of L-GA determined for A1.3) is more than the amount determined for A2.3), then the variant to be tested displays activity in Assay A.

If the amount of butyl-phosphoester of L-GA determined for A1.3) is the same or less than the amount determined for A2.3), then the variant to be tested does not display activity in Assay A.

Preferable formate solutions in steps A1.3) and A2.3) are ammonium formate or sodium formate solutions. Alternatively, the reaction in steps A 1.3) and A2.3) can also be stopped by adding methanol, preferably 1 ml of methanol.

4.3.3.3.2 Assay B

In a further assay (“Assay B”), the activity of a polypeptide variants of any of SEQ ID NO: 2, SEQ ID NO: 6, SEQ ID NO: 19, SEQ ID NO: 22, SEQ ID NO: 23, SEQ ID NO: 10, SEQ ID NO: 14, SEQ ID NO: 18, SEQ ID NO: 20, SEQ ID NO: 24, SEQ ID NO: 25, SEQ ID NO: 26, SEQ ID NO: 27, SEQ ID NO: 28, SEQ ID NO: 34, SEQ ID NO: 17, SEQ ID NO: 21, SEQ ID NO: 29, SEQ ID NO: 30, SEQ ID NO: 31, SEQ ID NO: 32, and SEQ ID NO: 33, respectively, with respect to the polypeptide may be determined.

Assay B is carried out by the following steps:
B1) First, the activity of the “standard” polypeptide standard (i.e., one polypeptide sequence selected from SEQ ID NO: 2, SEQ ID NO: 6, SEQ ID NO: 19, SEQ ID NO: 22, SEQ ID NO: 23, SEQ ID NO: 10, SEQ ID NO: 14, SEQ ID NO: 18, SEQ ID NO: 20, SEQ ID NO: 24, SEQ ID NO: 25, SEQ ID NO: 26, SEQ ID NO: 27, SEQ ID NO: 28, SEQ ID NO: 34, SEQ ID NO: 17, SEQ ID NO: 21, SEQ ID NO: 29, SEQ ID NO: 30, SEQ ID NO: 31, SEQ ID NO: 32, and SEQ ID NO: 33) is determined by the following steps B1.1), B1.2), and B1.3) as follows:
B1.1) 990 μl of a reaction solution containing phosphate buffer (0.1 M, pH 7.5), 3 mM O-acetyl L-homoserine-HCl, 10 μM pyridoxal 5-phosphate mono-hydrate and 1.0 nmol of the “standard” polypeptide to be tested are prepared and heated to 50° C.
B1.2) The reaction is started by adding 10 μl of a 200 mM solution of butyl-P-methylphosphinate (MPBE, CAS #6172-80-1). The concentration of MPBE in the reaction solution is 2 mM.
B1.3) After addition of the MPBE, the reaction is conducted for 120 minutes at 50° C. Then, the reaction is stopped by adding 10 μl 1% formate solution and cooling on ice.
B2) Then, steps B1.1), B1.2), and B1.3) are repeated with the variant: B2.1) 990 μl of a reaction solution containing phosphate buffer (0.1 M, pH 7.5), 3 mM O-acetyl L-homoserine-HCl, 10 μM pyridoxal 5-phosphate mono-hydrate and 1.0 nmol of the “variant” polypeptide to be tested are prepared and heated to 50° C.
B2.2) The reaction is started by adding 10 μl of a 200 mM solution of butyl-P-methylphosphinate (MPBE, CAS #6172-80-1). The concentration of MPBE in the reaction solution is 2 mM.
B2.3) After addition of the MPBE, the reaction is conducted for 120 minutes at 50° C. Then, the reaction is stopped by adding 10 μl 1% formate solution and cooling on ice.
B3) Finally, the amount of the butyl-phosphoester of L-GA (i.e. the compound according to formula (III) with R¹=n-butyl) obtained in the reaction solution in B1.3) and B2.3) is determined and compared, preferably by LC-MS analysis described under item 5.7.
B4) Then, the ratio of the amount (in mole) of butyl-phosphoester of L-GA obtained in B2.3) is divided by the amount (in mole) of butyl-phosphoester of L-GA obtained in B1.3). This ratio is then multiplied with a factor of 100, giving the relative activity of the variant polypeptide with respect to the “standard” polypeptide in percent.

Preferable formate solutions in steps B1.3) and B2.3) are ammonium formate or sodium formate solutions. Alternatively, the reaction in steps B1.3) and B2.3) can also be stopped by adding methanol, preferably 1 ml of methanol.

4.3.3.4 “Preferred Variants”

4.3.3.4.1 “Variants of SEQ ID NO: 2”

In particular, a variant of SEQ ID NO: 2 is a polypeptide with sequence identity of ≥80%, more preferably a ≥85%, more preferably a ≥90%, more preferably ≥91%, more preferably ≥92%, more preferably a ≥93%, more preferably a ≥94%, more preferably ≥95%, more preferably ≥96%, more preferably a ≥97%, more preferably a ≥98%, more preferably ≥99%, more preferably ≥99.9% sequence identity to polypeptide sequence SEQ ID NO: 2.

Preferred variants of SEQ ID NO: 2 display activity in Assay A under item 4.3.3.3.1.

Even more preferably, the activity of the respective variant of SEQ ID NO: 2 is at least 1%, preferably at least 10%, more preferably at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99% relative to the activity of SEQ ID NO: 2 as determined in Assay B under item 4.3.3.3.2.

Even more preferably, the activity of the respective variant of SEQ ID NO: 2 is in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% relative to the activity of SEQ ID NO: 2 as determined in Assay B under item 4.3.3.3.2.

4.3.3.4.2 “Variants of SEQ ID NO: 6”

In particular, a variant of SEQ ID NO: 6 is a polypeptide with sequence identity of ≥80%, more preferably a ≥85%, more preferably a ≥90%, more preferably a ≥91%, more preferably a ≥92%, more preferably a ≥93%, more preferably a ≥94%, more preferably a ≥95%, more preferably a ≥96%, more preferably a ≥97%, more preferably a ≥98%, more preferably a ≥99%, more preferably a ≥99.9% sequence identity to polypeptide sequence SEQ ID NO: 6.

Preferred variants of SEQ ID NO: 6 display activity in Assay A under item 4.3.3.3.1.

Even more preferably, the activity of the respective variant of SEQ ID NO: 6 is at least 1%, preferably at least 10%, more preferably at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99% relative to the activity of SEQ ID NO: 6 as determined in Assay B under item 4.3.3.3.2.

Even more preferably, the activity of the respective variant of SEQ ID NO: 6 is in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% relative to the activity of SEQ ID NO: 6 as determined in Assay B under item 4.3.3.3.2.

4.3.3.4.3 “Variants of SEQ ID NO: 10”

In particular, a variant of SEQ ID NO: 10 is a polypeptide with sequence identity of ≥80%, more preferably a ≥85%, more preferably a ≥90%, more preferably a ≥91%, more preferably a ≥92%, more preferably a ≥93%, more preferably a ≥94%, more preferably a ≥95%, more preferably a ≥96%, more preferably a ≥97%, more preferably a ≥98%, more preferably a ≥99%, more preferably a ≥99.9% sequence identity to polypeptide sequence SEQ ID NO: 10.

Preferred variants of SEQ ID NO: 10 show activity in Assay A under item 4.3.3.3.1.

Even more preferably, the activity of the respective variant of SEQ ID NO: 10 is at least 1%, preferably at least 10%, more preferably at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99% relative to the activity of SEQ ID NO: 10 as determined in Assay B under item 4.3.3.3.2.

Even more preferably, the activity of the respective variant of SEQ ID NO: 10 is in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% relative to the activity of SEQ ID NO: 10 as determined in Assay B under item 4.3.3.3.2.

4.3.3.4.4 “Variants of SEQ ID NO: 14”

In particular, a variant of SEQ ID NO: 14 is a polypeptide with sequence identity of ≥80%, more preferably a ≥85%, more preferably a ≥90%, more preferably a ≥91%, more preferably a ≥92%, more preferably a ≥93%, more preferably a ≥94%, more preferably a ≥95%, more preferably a ≥96%, more preferably a ≥97%, more preferably a ≥98%, more preferably a ≥99%, more preferably a ≥99.9% sequence identity to polypeptide sequence SEQ ID NO: 14.

Preferred variants of SEQ ID NO: 14 display activity in Assay A under item 4.3.3.3.1.

Even more preferably, the activity of the respective variant of SEQ ID NO: 14 is at least 1%, preferably at least 10%, more preferably at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99% relative to the activity of SEQ ID NO: 14 as determined in Assay B under item 4.3.3.3.2.

Even more preferably, the activity of the respective variant of SEQ ID NO: 14 is in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% relative to the activity of SEQ ID NO: 14 as determined in Assay B under item 4.3.3.3.2.

4.3.3.4.5 Further Variants

Preferred variants of any of SEQ ID NO: 19, SEQ ID NO: 22, SEQ ID NO: 23, SEQ ID NO: 18, SEQ ID NO: 20, SEQ ID NO: 24, SEQ ID NO: 25, SEQ ID NO: 26, SEQ ID NO: 27, SEQ ID NO: 28, SEQ ID NO: 34, SEQ ID NO: 17, SEQ ID NO: 21, SEQ ID NO: 29, SEQ ID NO: 30, SEQ ID NO: 31, SEQ ID NO: 32, SEQ ID NO: 33, respectively, can be determined mutatis mutandis as described for SEQ ID NO: 2 as described under item 4.3.3.4.1.

Preferred variants of SEQ ID NO: 19 can be determined by the procedure that results when in the description under item 4.3.3.4.1, the word “SEQ ID NO: 2” is substituted by the word “SEQ ID NO: 19”.

Preferred variants of SEQ ID NO: 22 can be determined by the procedure that results when in the description under item 4.3.3.4.1, the word “SEQ ID NO: 2” is substituted by the word “SEQ ID NO: 22”.

Preferred variants of SEQ ID NO: 23 can be determined by the procedure that results when in the description under item 4.3.3.4.1, the word “SEQ ID NO: 2” is substituted by the word “SEQ ID NO: 23”.

Preferred variants of SEQ ID NO: 18 can be determined by the procedure that results when in the description under item 4.3.3.4.1, the word “SEQ ID NO: 2” is substituted by the word “SEQ ID NO: 18”.

Preferred variants of SEQ ID NO: 20 can be determined by the procedure that results when in the description under item 4.3.3.4.1, the word “SEQ ID NO: 2” is substituted by the word “SEQ ID NO: 20”.

Preferred variants of SEQ ID NO: 24 can be determined by the procedure that results when in the description under item 4.3.3.4.1, the word “SEQ ID NO: 2” is substituted by the word “SEQ ID NO: 24”.

Preferred variants of SEQ ID NO: 25 can be determined by the procedure that results when in the description under item 4.3.3.4.1, the word “SEQ ID NO: 2” is substituted by the word “SEQ ID NO: 25”.

Preferred variants of SEQ ID NO: 26 can be determined by the procedure that results when in the description under item 4.3.3.4.1, the word “SEQ ID NO: 2” is substituted by the word “SEQ ID NO: 26”.

Preferred variants of SEQ ID NO: 27 can be determined by the procedure that results when in the description under item 4.3.3.4.1, the word “SEQ ID NO: 2” is substituted by the word “SEQ ID NO: 27”.

Preferred variants of SEQ ID NO: 28 can be determined by the procedure that results when in the description under item 4.3.3.4.1, the word “SEQ ID NO: 2” is substituted by the word “SEQ ID NO: 28”.

Preferred variants of SEQ ID NO: 34 can be determined by the procedure that results when in the description under item 4.3.3.4.1, the word “SEQ ID NO: 2” is substituted by the word “SEQ ID NO: 34”.

Preferred variants of SEQ ID NO: 17 can be determined by the procedure that results when in the description under item 4.3.3.4.1, the word “SEQ ID NO: 2” is substituted by the word “SEQ ID NO: 17”.

Preferred variants of SEQ ID NO: 21 can be determined by the procedure that results when in the description under item 4.3.3.4.1, the word “SEQ ID NO: 2” is substituted by the word “SEQ ID NO: 21”.

Preferred variants of SEQ ID NO: 29 can be determined by the procedure that results when in the description under item 4.3.3.4.1, the word “SEQ ID NO: 2” is substituted by the word “SEQ ID NO: 29”.

Preferred variants of SEQ ID NO: 30 can be determined by the procedure that results when in the description under item 4.3.3.4.1, the word “SEQ ID NO: 2” is substituted by the word “SEQ ID NO: 30”.

Preferred variants of SEQ ID NO: 31 can be determined by the procedure that results when in the description under item 4.3.3.4.1, the word “SEQ ID NO: 2” is substituted by the word “SEQ ID NO: 31”.

Preferred variants of SEQ ID NO: 32 can be determined by the procedure that results when in the description under item 4.3.3.4.1, the word “SEQ ID NO: 2” is substituted by the word “SEQ ID NO: 32”.

Preferred variants of SEQ ID NO: 33 can be determined by the procedure that results when in the description under item 4.3.3.4.1, the word “SEQ ID NO: 2” is substituted by the word “SEQ ID NO: 33”.

### 4.4 Method Conditions

The reaction in step a) of the method according to the present invention may be carried out under conditions known to the skilled person.

The reaction medium in which activated L-homoserine H_{A} is reacted with the substrate S is preferably aqueous, more preferably an aqueous buffer.

Exemplary buffers commonly used in biotransformation reactions and advantageously used herein include Tris, phosphate, or any of Good's buffers, such as 2-(N-morpholino)ethanesulfonic acid (“MES”), N-(2-acetamido)iminodiacetic acid (“ADA”), piperazine-N,N′-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethy)-2-aminoethanesulfonic acid (“BES”), 2-[[1,3-dihydroxy-2-(hydroxymethyopropan-2-yl]amino]ethanesulfonic acid (“TES”), 4-(2-hydroxyethy)-1-piperazineethanesulfonic acid (“HEPES”), 3-(Bis(2-hydroxyethyoamino)-2-hydroxypropane-1-sulfonic acid (“DIPSO”), acetamidoglycine, 3-(N-Tris(hydroxymethyomethylamino(-2-hydroxypropane)sulfonic acid (“TAPSO”), piperazine-N,N′-bis(2-hydroxypropanesulfonic acid) (“POPSO”), 4-(2-Hydroxyethyopiperazine-1-(2-hydroxypropanesulfonic acid) (“HEPPSO”), 3-[4-(2-HydroxyethyD)-1-piperazinyl]propanesulfonic acid (“HEPPS”), tricine, glycinamide, bicine, or 3-[[1,3-dihydroxy-2-(hydroxymethylpropan-2-yl]amino]propane-1-sulfonic acid (“TAPS”).

In some embodiments, ammonium can act as a buffer. One or more organic solvents can also be added to the reaction.

Preferably, step a) of the method according to the invention is carried out in a phosphate buffer.

The pH of the reaction medium in step a) of the method is preferably in the range of from 2 to 10, more preferably in the range of from 5 to 8, most preferably 7.5.

The method according to the invention is preferably carried out at a temperature in the range of from 20° C. to 70° C., more preferably in the range of from 30° C. to 55° C., most preferably 50° C.

### 4.5 L-Glufosinate Phosphoester

The product of the method according to the invention, is a compound of the following structure (III):

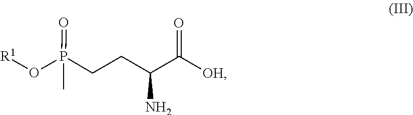

The compound according to structure (III) is L-glufosinate (for R¹=H) or L-glufosinate phosphoester (for R¹=alkyl, alkenyl, alkinyl, hydroxyalkyl or aryl).

The compound according to structure (III), wherein R¹=n-butyl, is abbreviated as “L-GA-Bu” or “LGA-Bu”.

The skilled person understands that the identity of residue R¹ in the compound according to structure (III) depends on the identity of residue R¹ in the substrate structure (I).

If R¹ is hydrogen, L-glufosinate is directly obtained in the method according to the invention.

In the preferred embodiment, in which R¹ is selected from alkyl, alkenyl, alkinyl, hydroxyalkyl, aryl, preferably alkyl, more preferably methyl, ethyl, n-butyl, the compound (III) is a L-glufosinate phosphoester.

In those embodiments, it is further preferable that the method according to the invention contains a further step b) wherein the compound of the structure (III) which is obtained in step (a), and in which R¹ is selected from alkyl, alkenyl, alkinyl, hydroxyalkyl, aryl, preferably alkyl, more preferably methyl, ethyl, n-butyl, is saponified to give L-glufosinate.

This can be carried out by methods known to the skilled person.

Preferably, such saponification is carried out under acidic conditions, more preferably by mixing 1 Volume of the reaction medium containing the compound of the structure (III) which is obtained in step (a) and 4 Volumes of 6N HCl for 2 h and incubating the resulting mixture at a temperature of 50° C. to 150° C., preferably at 100° C.

Alternatively, an enzymatic saponification can be carried out.

### 5. EXAMPLES

Genes of different origin encoding sulfhydrylase (EC 2.5.1.- and EC 2.5.1.49) were tested for their ability to react with an activated homoserine derivate and different substrates according to structure (I) to form a glufosinate derivate.

### 5.1 Example 1 Identification of Suitable Enzymes and Construction of Plasmids

### 5.1.1 Examined Sulfhydrylase

Bibliographic details of the genes of O-acetylhomoserine sulfhydrylases and O-succinylhomoserine sulfhydrylases that were used in the examples are summarized in table 2.

TABLE 2

| | | | | ReferenceNCBI | SEQ |
|---|---|---|---|---|---|
| Strain | comment | Gene name | Gene ID | accession | ID NO: |
| Corynebacterium | wild type | metY | NCgl0625 | NC_003450 | 1 |
| glutamicum | | | | | |
| ATCC 13032 | | | | | |
| Corynebacterium | metY variant | metY_P2T | | | 5 |
| glutamicum | | | | | |
| ATCC 13032 | | | | | |
| Chromobacterium | wild type | metZ | CV_2725 | AE016825 | 9 |
| violaceum | | | | | |
| ATCC 12472 | | | | | |
| Hyphomonas | wild type | metZ | HNE_2672 | CP000158 | 13 |
| neptunium | | | | | |
| ATCC 15444 | | | | | |

### 5.1.2 Preparation of the Gene Expression Plasmids

5.1.2.1 O-acetylhomoserine sulfhydrylase metY derived from Corynebacterium glutamicum

The metY gene derived from Corynebacterium glutamicum ATCC 13032 codes for an O-acetylhomoserine sulfhydrylase and can be found at the NCBI under locus tag NCgl0625 of the genome sequence accessible under NC_003450.

To realize the expression of the enzyme, the expression vector pET-26b(+) (Novagen EMD Millipore) was used. Therefore, the metY_Cg polynucleotide according to SEQ ID NO: 3 was synthesized by GeneArt (ThermoFisher Scientific (Waltham, USA)).

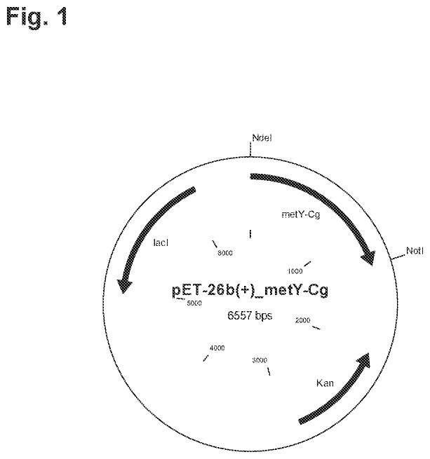

To assemble the expression vector pET-26b(+)_metY-Cg, the vector pET-26(+) and the metY_Cg polynucleotide were both treated with NdeI and NotI, ligated, and the ligation mixture was used to transform E. coli. The resulting vector, pET-26b(+)_metY-Cg, is shown schematically in FIG. 1. DNA of the expression vector pET-26b(+)_metY-Cg was isolated from a transformant.

5.1.2.2 O-acetylhomoserine sulfhydrylase metY_P2T derived from Corynebacterium glutamicum

The O-acetylhomoserine sulfhydrylase variant metY_P2T is encoded by a polynucleotide according to SEQ ID NO: 5, which is identical to the polynucleotide of SEQ ID NO: 1, except that the nucleobases from position 4 to 6 are aca.

The polynucleotide of SEQ ID NO: 5 encodes a polypeptide comprising the amino acid sequence of SEQ ID NO: 6, which is identical to the polynucleotide of SEQ ID NO: 1, except that the amino acid proline at position 2 is substituted by threonine.

To realize the expression of the enzyme variant metY_P2T, the expression vector pET-26b(+) (Novagen EMD Millipore) was used. Therefore, the metY_P2T polynucleotide according to SEQ ID NO: 7 was synthesized by GeneArt (ThermoFisher Scientific (Waltham, USA)).

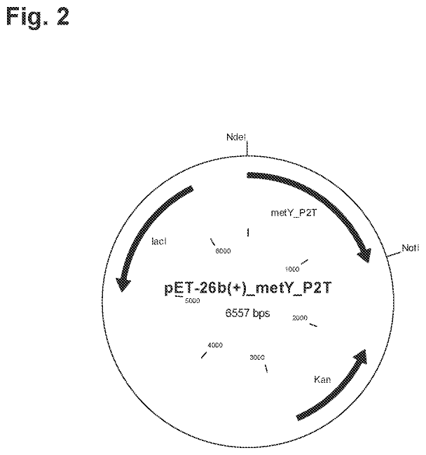

To assemble the expression vector pET-26b(+)_metY_P2T, the vector pET-26(+) and the metY_P2T polynucleotide were both treated with NdeI and NotI, ligated, and the ligation mixture was used to transform E. coli. The resulting vector, pET-26b(+)_metY_P2T, is shown schematically in FIG. 2.

DNA of the expression vector pET-26b(+)_metY_P2T was isolated from a transformant.

5.1.2.3 O-succinylhomoserine sulfhydrylase metZ derived from Chromobacterium violaceaum

The metZ gene derived from Chromobacterium violaceaum ATCC 12472 codes for an O-succinylhomoserine sulfhydrylase and can be found at the NCBI under locus tag CV_2725 of the genome sequence accessible under AE016825.

To realize the expression of the enzyme, the expression vector pET-26b(+) (Novagen EMD Millipore) was used. Therefore, the metZ_Cv polynucleotide according to SEQ ID NO: 11 was synthesized by GeneArt (ThermoFisher Scientific (Waltham, USA)).

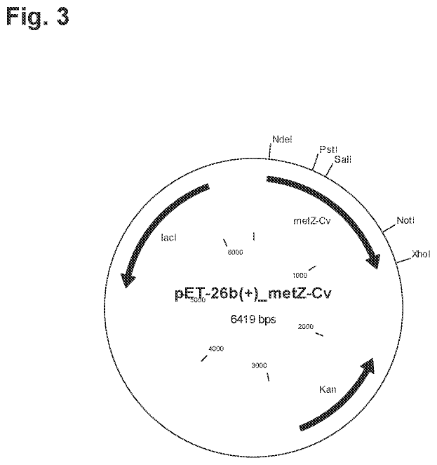

To assemble the expression vector pET-26b(+)_metZ-Cv the vector pET-26(+) and the metZ_Cv polynucleotide were both treated with NdeI and XhoI, ligated, and the ligation mixture was used to transform E. coli. The resulting vector, pET-26b(+)_metZ-Cv, is shown schematically in FIG. 3. DNA of the expression vector pET-26b(+)_metZ-Cv was isolated from a transformant.

5.1.2.4 O-succinylhomoserine sulfhydrylase metZ derived from Hyphomonas neptunium

The metZ gene derived from Hyphomonas neptunium ATCC 15444 codes for an O-succinylhomoserine sulfhydrylase and can be found at the NCBI under locus tag HNE_2672 of the genome sequence accessible under CP000158.

To realize the expression of the enzyme, the expression vector pET-26b(+) (Novagen EMD Millipore) was used. Therefore, the metZ_Hn polynucleotide according to SEQ ID NO: 15 was synthesized by GeneArt (ThermoFisher Scientific (Waltham, USA)).

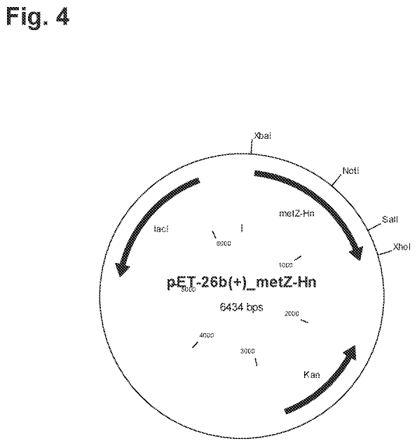

To assemble the expression vector pET-26b(+)_metZ-Hn, the vector pET-26(+) and the metZ_Hn polynucleotide were both treated with XbaI and XhoI, ligated, and the ligation mixture was used to transform E. coli. The resulting vector, pET-26b(+)_metZ-Hn, is shown schematically in FIG. 4.

DNA of the expression vector pET-26b(+)_metZ-Hn was isolated from a transformant.

### 5.2 Transforming and Expression of the Sulfhydrylase

These vectors carrying the sulfhydrylases genes were each transformed in Escherichia coli BL21 (DE3) (New England Biolabs), which were subsequently cultured on LB medium agar plates with 50 mg/l kanamycin at 37° C. for 16 h. Strain BL21 carrying the vector pET26b(+) without any insert was used as negative control.

The resulting strain were named Ec BL21 pET-26b(+)_metY-Cg, Ec BL21 pET-26b(+)_metY-Cg_P2T, Ec BL21 pET-26b(+)_metZ-Cv, Ec BL21 pET-26b(+)_metZ-Hn and Ec BL21 pET-26b(+) respectively.

In each case a colony has been selected which was inoculated into 10 ml of LB medium with 50 mg/l kanamycin and cultured at 37° C., 250 rpm for 6 hours. 50 μl of LB medium were subsequently treated with 50 mg/l kanamycin and inoculated with 50 μl of the growth cell culture and incubated at 28° C., 250 rpm for 16 h. This cell culture was diluted with 200 mi of fresh LB medium containing 50 μg/l kanamycin in a 2 l flask to an OD of 0.15 and was further cultured under identical conditions until an OD of 0.5 was attained (circa 4 h). The start point of the induction of gene expression was then affected by adding 200 μl of a 300 mM IPTG stock solution (final concentration 300 μM isopropyl-β-D-thiogalactopyranoside (IPTG), Sigma-Aldrich, Germany). The induction was carried out at 28° C., 250 rpm for 4 h. The culture was then harvested (8 ml normalised to an OD=1), the supernatant removed by centrifugation (20 min, 4000 rpm, 4° C.) and the pelleted cells were washed twice with 800 μl of 0.1 M potassium phosphate buffer (pH 7.5) and taken up in 1 mi of buffer. The mechanical cell digestion was carried out in a FastPrep FP120 instrument (QBiogene, Heidelberg), wherein the cells were shaken four times for 30 s at 6.5 m/s in digestion vessels with 300 mg of glass beads (Ø0.2-0.3 mm). The crude extract was then centrifuged at 12000 rpm, 4° C., 20 min, in order to remove undigested cells and cell debris. The supernatant was used for the enzyme assay. The concentration of the polypeptide in the supernatant was determined by SDS page and analysis of the respective bands via the software GelQuant® (BiochemLabSolutions).

### 5.3 Inventive Examples I1 to I4

The following assay was carried out to determine whether the respective polypeptide catalyzed the reaction of the respective phosphor-containing substrate and activated L-homoserine. O-acetyl L-homoserine was used as activated L-homoserine substrate.

To 880 μl phosphate buffer (0.1 M, pH 7.5) containing 1 nmol of the polypeptide to be tested were added 100 μl of a 30 mM aqueous solution of 0-acetyl homoserine-HCl, 10 μl of a 1 mM aqueous solution of pyridoxal 5′-phosphate mono-hydrate (1 mM), and 10 μl of a 200 mM aqueous solution of Butyl P-methylphosphinate (CAS-No. 6172-80-1; “MPBE”). The reaction was conducted for 120 min at 50° C. Then, 100 μl batch solution was diluted in 100 μl methanol and applied on the LC-MS QQQ (item 5.7) to analyse the LGA-Bu.

### 5.4 Inventive Examole I5

Catalysis of the polypeptide isolated from pET-26b(+)_metY-Cg_P2T was further tested in a reaction with methyl P-methylphosphinate (“MPME”; CAS-No. 16391-06-3).

To 880 μl phosphate buffer (0.1 M, pH 7.5) containing 1 nmol of this polypeptide were added 100 μl of a 30 mM aqueous solution of 0-acetyl homoserine-HCl, 10 μl of a 1 mM aqueous solution of pyridoxal 5′-phosphate mono-hydrate (1 mM), and 10 μl of a 200 mM aqueous solution of MPME. The reaction was conducted for 120 min at 50° C. Then, 100 μl batch solution was diluted in 100 μl methanol and applied on the LC-MS QQQ (item 5.7) to analyse the LGA-Bu.

### 5.4 Comparative Examples C1 to C4

It was further investigated whether other phosphor compounds would serve as substrates for the above reaction. To this end, diethyl methylphosphonite (“DEMP”; CAS-No. 15715-41-0) was tested.

To 880 μl phosphate buffer (0.1 M, pH 7.5) containing 1 nmol of the polypeptide to be tested were added 100 μl of a 30 mM aqueous solution of 0-acetyl homoserine-HCl, 10 μl of a 1 mM aqueous solution of pyridoxal 5′-phosphate mono-hydrate (1 mM), and 10 μl of a 200 mM aqueous solution of DEMP. The reaction was conducted for 120 min at 50° C. Then, 100 μl batch solution was diluted in 100 μl methanol and applied on the LC-MS QQQ (item 5.7) to analyse the LGA-Bu.

### 5.5 Comparative Examples C5 to C8

It was further investigated whether the respective polypeptide would catalyze the wild type reaction of O-acetylhomoserine with methylmercaptane (“MM”; CAS-No. 74-93-1) according to the above reaction <1A>.

To 880 μl phosphate buffer (0.1 M, pH 7.5) containing 1 nmol of the polypeptide to be tested were added 100 μl of a 30 mM aqueous solution of 0-acetyl homoserine-HCl, 10 μl of a 1 mM aqueous solution of pyridoxal 5′-phosphate mono-hydrate (1 mM), and 10 μl of a 200 mM aqueous solution of MM. The reaction was conducted for 120 min at 50° C. Then, 100 μl batch solution was diluted 40 in 100 μl methanol and applied on the LC-MS QQQ (item 5.7) to analyse for the respective product.

In case of inventive examples 11 to 14, the product is the compound according to structure (III), wherein R¹=n-butyl (“LGA-Bu”).

In case of inventive example 15, the product is the compound according to structure (III), wherein R¹=methyl.

In case of comparative examples C1 to C4, the product is the compound according to structure (III), wherein R¹=ethyl (“LGA-Et”).

In case of comparative examples C5 to C6, the product is methionine.

The results are summarized in the following table 3. The abbreviations used are
“DEMP”=methyl diethoxy phosphine (CAS-No. 15715-41-0);
“MPBE”=butyl P-methylphosphinate (CAS-No. 6172-80-1);
“MPME”=methyl P-methylphosphinate (CAS-No. 16391-06-3);
“MM”=methyl mercaptane (CAS-No: 74-93-1).

TABLE 3

| Example | Enzyme | Polypeptide | Substrate | Product |
|---|---|---|---|---|
| I1 | Cg_MetY | SEQ ID NO: 2 | MPBE | yes |
| I2 | Cg_MetY_P2T | SEQ ID NO: 6 | MPBE | yes |
| I3 | Cv_MetZ | SEQ ID NO: 10 | MPBE | yes |
| I4 | Hn_MetZ | SEQ ID NO: 14 | MPBE | yes |
| I5 | Cg_MetY_P2T | SEQ ID NO: 6 | MPME | yes |
| C1 | Cg_MetY | SEQ ID NO: 2 | DEMP | no |
| C2 | Cg_MetY_P2T | SEQ ID NO: 6 | DEMP | no |
| C3 | Cv_MetZ | SEQ ID NO: 10 | DEMP | no |
| C4 | Hn_MetZ | SEQ ID NO: 14 | DEMP | no |
| C5 | Cg_MetY | SEQ ID NO: 2 | MM | yes |
| C6 | Cg_MetY_P2T | SEQ ID NO: 6 | MM | yes |
| C7 | Cv_MetZ | SEQ ID NO: 10 | MM | yes |
| C8 | Hn_MetZ | SEQ ID NO: 14 | MM | yes |

### 5.6 Results

The results summarized in table 3 surprisingly show that the tested polypeptides accept other substrates methylphosphinic acid compounds such as MPBE and MPME, and catalyze their reaction with activated L-homoserine to the respective n-butyl or methyl phosphate ester of LGA. This finding was even more surprising, as the skilled person would not have expected that these enzyme would catalyze these reactions, as other phosphate compounds such as DEMP do not work as substrates.

This finding opens new enzymatic pathways to LGA and its derivatives.

### 5.7 Analytical Methods

All analytical measurements for the experiments have been carried out via a scan on a LC-MS QQQ system. Samples were diluted in in methanol (v:v=1:2).

The applicated HPLC belongs to the 1260 Infinity-series from Agilent connected to a mass spectrometer 6420 triple quadrupole with electrospray ionization. Peak identification was carried out via retention time and molecular mass in a positive detection mode.

Data evaluation was carried out via peak area and a quadratic calibration without zero.

Acquisition Method Details

Ion Source ESI (electrospray ionization)

Time Parameters

Mass Range m/z=50-300

Scans per second 400

Fragmentation 40 V

Polarity Positive (Scan-Mode)

Source Parameters

Gas temperature 350° C.

Gas flow 12 l/min

Nebulizer 50 psi

Capillary 4000 V

Binary Pump

Injection Volume 2.00 μL

Flow 0.60 mL/min

Solvent Composition

Solvent A 100 mM Ammonium acetate plus 0.1% (v/v) formic acid in H₂O

Solvent B 0.1% formic acid in acetonitrile

Gradient see table 4.

TABLE 4

| Time (min) | Solvent A (%) | Solvent B (%) |
|---|---|---|
| 0.5 | 5 | 95 |
| 1.2 | 45 | 55 |
| 4.0 | 45 | 55 |
| 4.1 | 95 | 5 |
| 7.0 | 95 | 5 |
| 7.1 | 5 | 95 |
| 10.0 | 5 | 95 |

Column

Type Luna HILIC; 100×2 mm; 3 μm; Phenomenex 00D-4449-BO

Temperature 30.0° C.

### 6. SEQUENCE OVERVIEW

The following table provides an overview of the DNA and protein sequences referred to in the context of the present application:

Abbreviations used are: “AHS”=-acetylhomoserine sulfhydrylase; “SHS”=O-succinylhomoserine sulfhydrylase; “CGS”=cystathionine γ-synthase.

| SEQ | |
|---|---|
| ID NO: | |
| 1 | DNA sequence of metY gene (nucleotide sequence comprising locus_tag NCgl0625 disclosed in NC_003450) from Corynebacterium |
| | glutamicum ATCC 13032, encoding AHS. |
| 2 | Protein sequence translated from SEQ ID NO: 1. |
| 3 | DNA sequence: nucleotides 7 to 1320 are identical with SEQ ID NO: 1, further containing six additional nucleotides (aaacat) at position 1 |
| | to 6, thus forming a Ndel restriction site (nucleotides 4 to 9), and ten additional nucleotides at 1321-1330 (gcggccgcaaa), forming a Notl |
| | restriction site (nucleotides 1321-1328). |
| 4 | Protein sequence translated from nucleotides 7 to 1320 of SEQ ID NO: 3; identical with SEQ ID NO: 2. |
| 5 | DNA sequence identical to SEQ ID NO: 1, except for one allelic variation [cca (for Pro)→ aca (for Thr)] at nucleotides 4-6. “metY_P2T”- |
| | variant of AHS |
| 6 | Protein sequence translated from SEQ ID NO: 5 (99.8% sequence identity with SEQ ID NO: 2). |
| 7 | DNA sequence: nucleotides 7 to 1320 are identical with SEQ ID NO: 5, further containing six additional nucleotides (aaacat) at position 1 |
| | to 6, thus forming a Ndel restriction site (nucleotdies 4 to 9), and ten additional nucleotides at 1321-1330 (gcggccgcaaa), forming a Notl |
| | restriction site. |
| 8 | Protein sequence translated from nucleotides 7 to 1320 of SEQ ID NO: 7; identical with SEQ ID NO: 6. |
| 9 | DNA sequence of metZ gene (nucleotide sequence comprising locus_tag CV_2725 disclosed in AE016825) from Chromobacterium |
| | violaceaum ATCC 12472, encoding SHS. |
| 10 | Protein sequence translated from SEQ ID NO: 9. |
| 11 | DNA sequence: nucleotides 7 to 1191 are identical with SEQ ID NO: 9, further containing six additional nucleotides (aaacat) at position 1 |
| | to 6, thus forming a Ndel restriction site (nucleotides 4 to 9), and nine additional nucleotides at positions 1192-1200 (ctcgagaaa), forming |
| | a Xhol restriction site (nucleotides 1192-1197). |
| 12 | Protein sequence translated from nucleotides 7 to 1191 of SEQ ID NO: 9; identical with SEQ ID NO: 10. |
| 13 | DNA sequence of metZ gene (nucleotide sequence comprising locus_tag HNE_2672 disclosed in CP000158) from Hyphomonas |
| | neptunium ATCC 15444, encoding SHS. |
| 14 | Protein sequence translated from SEQ ID NO: 13. |
| 15 | DNA sequence: nucleotides 46 to 1245 are identical with SEQ ID NO: 13, further containing 45 additional nucleotides (aaatctagaa |
| | ataattttgt ttaactttaa gaaggagata tacat) at position 1 to 45, thus forming a Xbal restriction site (nucleotides 4 to 9), and nine additional |
| | nucleotides at positions 1246-1254 (ctcgagaaa), forming a Xhol restriction site (nucleotides 1246-1251). |
| 16 | Protein sequence translated from nucleotides 46 to 1245 of SEQ ID NO: 13; identical with SEQ ID NO: 14. |
| 17 | Protein sequence of metB-gene from Escherichia coli K-12, encoding CGS. |
| 18 | Protein sequence of metZ-gene from Pseudomonas aeruginosa PAO1, encoding SHS. |
| 19 | Protein sequence of metY-gene from Pseudomonas aeruginosa PAO1, encoding AHS. |
| 20 | Protein sequence of metZ-gene from Pseudomonas putida KT2440, encoding SHS. |
| 21 | Protein sequence of metB-gene from Corynebacterium glutamicum ATCC 13032, encoding CGS. |
| 22 | Protein sequence of met17 gene from Leptospira interrogans serovar Lai str. 56601, encoding AHS. |
| 23 | Protein sequence of Met17 gene from Saccharomyces cerevisiae S288c, encoding AHS. |
| 24 | Protein sequence of metZ-gene from Nocardia farcinica IFM 10152, encoding SHS. |
| 25 | Protein sequence of metZ-gene from Bradyrhizobium japonicum USDA 6, encoding SHS. |
| 26 | Protein sequence of metZ-gene from Methylococcus capsulatus str. Bath, encoding SHS. |
| 27 | Protein sequence from Methylobacillus flagellatus KT, encoding an SHS, therefore abbreviated herein as “metZ”. |
| 28 | Protein sequence of metZ-gene from Nitrosomonas europaea ATCC 19718, encoding SHS. |
| 29 | Protein sequence of metB-gene from Klebsiella pneumoniae subsp. pneumoniae MGH 78578, encoding CGS. |
| 30 | Protein sequence of metI-gene from Bacillus subtilis subsp. subtilis str. 168, encoding CGS. |
| 31 | Protein sequence of metB-gene from Shigella flexneri 2a str. 2457T, encoding CGS. |
| 32 | Protein sequence of metB-gene from Colwellia psychrerythraea 34H, encoding CGS. |
| 33 | Protein sequence of metB-gene from Salmonella enterica serovar Paratyphi A ATCC 9150, encoding CGS. |
| 34 | Protein sequence of metZ-gene from Rhodobacter sphaeroides ATCC 17029, encoding SHS. |

1. An enzymatically catalyzed method for producing L-glufosinate or a phosphoester thereof, the method comprising
(a) reacting an activated L-homoserine H_{A} with a substrate S of the following structure (I) to produce a compound of the following structure (III), 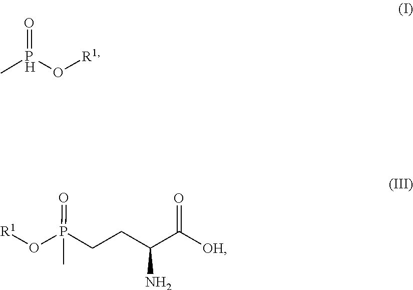
wherein R¹ is selected from the group consisting of hydrogen, an alkyl, an alkenyl, an alkinyl, a hydroxyalkyl, and an aryl, and
wherein the activated L-homoserine H_{A} has the following structure (II): 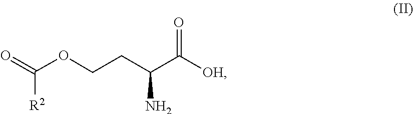
wherein R² is a hydrocarbon group with 1 to 15 carbon atoms which optionally comprises at least one functional group selected from OH, COOH, NH, and
wherein the reaction in (a) is catalyzed by at least one enzyme selected from the group consisting of a sulfhydrylase E₁ and a cystathionine γ-synthase E₂.

2. The method according to claim 1, wherein the activated L-homoserine H_{A} is selected from the group consisting of O-succinyl-L-homoserine and O-acetyl-L-homoserine.

3. The method according to claim 2, wherein the activated L-homoserine H_{A} is O-acetyl-L-homoserine.

4. The method according to claim 1, wherein R¹ is selected from the group consisting of hydrogen and an alkyl group.

5. The method according to claim 4, wherein the alkyl group is selected from the group consisting of methyl, ethyl, and n-butyl.

6. The method according to claim 1, wherein R¹ is selected from the group consisting of an alkyl, an alkenyl, an alkinyl, a hydroxyalkyl, and an aril, further comprising (b) saponifying the compound of the structure (II) obtained in (a) to give L-glufosinate.

7. The method according to claim 1, wherein the reaction in (a) is catalyzed by the sulfhydrylase E₁.

8. The method according to claim 7, wherein the sulfhydrylase E₁ is an O-acetyl homoserine sulfhydrylase or an O-succinyl homoserine sulfhydrylase.

9. The method according to claim 1, wherein the polypeptide sequence of the sulfhydrylase enzyme Et is selected from the group consisting of
SEQ ID NO: 2 and variants of SEQ ID NO: 2, SEQ ID NO: 6 and variants of SEQ ID NO: 6, SEQ ID NO: 19 and variants of SEQ ID NO: 19, SEQ ID NO: 22 and variants of SEQ ID NO: 22, SEQ ID NO: 23 and variants of SEQ ID NO: 23, SEQ ID NO: 10 and variants of SEQ ID NO: 10, SEQ ID NO: 14 and variants of SEQ ID NO: 14, SEQ ID NO: 18 and variants of SEQ ID NO: 18, SEQ ID NO: 20 and variants of SEQ ID NO: 20, SEQ ID NO: 24 and variants of SEQ ID NO: 24, SEQ ID NO: 25 and variants of SEQ ID NO: 25, SEQ ID NO: 26 and variants of SEQ ID NO: 26, SEQ ID NO: 27 and variants of SEQ ID NO: 27, SEQ ID NO: 28 and variants of SEQ ID NO: 28, and SEQ ID NO: 34 and variants of SEQ ID NO: 34,
and wherein the polypeptide sequence of the cystathionine γ-synthase E₂ is selected from the group consisting of SEQ ID NO: 17 and variants of SEQ ID NO: 17, SEQ ID NO: 21 and variants of SEQ ID NO: 21, SEQ ID NO: 29 and variants of SEQ ID NO: 29, SEQ ID NO: 30 and variants of SEQ ID NO: 30, SEQ ID NO: 31 and variants of SEQ ID NO: 31, SEQ ID NO: 32 and variants of SEQ ID NO: 32, and SEQ ID NO: 33 and variants of SEQ ID NO: 33, wherein variants are defined as polypeptide sequences with at least 80% sequence identity to the respective polypeptide sequence.

10. The method according to claim 1, wherein the activated L-homoserine H_{A} is prepared by fermentation of a strain producing activated L-homoserine H_{A}.

11. The method according to claim 10, wherein the strain producing activated L-homoserine H_{A} is selected from the group consisting of Escherichia sp., Erwinia sp., Serratia sp., Providencia sp., Corynebacterium sp., Pseudomonas sp., Leptospira sp., Salmonella sp., Brevibacterium sp., Hypomononas sp., Chromobacterium sp., and Norcardia sp., fungi.

12. The method according to claim 1, wherein R² is a hydrocarbon group with 1 to 15 carbon atoms comprising at least one functional group selected from the group consisting of OH, COOH, and NH.
